(19) United States
(12) Patent Application Publication
(10) Pub. No.: US 2025/0283046 A1
(43) Pub. Date: Sep. 11, 2025

(54) New Cell Populations and Means and Methods for their Differentiation and Preservation

(71) Applicant: EVOTEC INTERNATIONAL GMBH, Hamburg (DE)

(72) Inventors: Matthias AUSTEN, Hamburg (DE); Audrey HOLTZINGER, Hamburg (DE); Vanessa JANAS, Hamburg (DE); Saniye YUMLU, Hamburg (DE)

(21) Appl. No.: 18/858,555

(22) PCT Filed: Apr. 21, 2023
(86) PCT No.: PCT/EP2023/060467

§ 371 Date: Oct. 21, 2024

(30) Foreign Application Priority Data
Apr. 21, 2022 (EP) 22169238.7

(51) Int. Cl. C12N 5/071; C12N 5/00

(52) U.S. Cl. CPC C12N 5/0676; C12N 5/525; C12N 2501/115; C12N 2501/727; C12N 2501/734; C12N 2506/45

(57) ABSTRACT

The invention relates to the field of cell differentiation and cryopreservation, in particular of pancreatic lineage cells. It provides methods for differentiating cells of the pancreatic lineage, in particular to islet-like clusters. It further provides methods for freezing cells of the pancreatic lineage, in particular endocrine progenitor cells. It also provides new pancreatic lineage cell populations.

### FIELD OF THE INVENTION

The invention relates to the field of cell differentiation and cryopreservation, in particular of pancreatic lineage cells. It provides methods for differentiating cells of the pancreatic lineage, in particular to islet-like clusters. It further provides methods for freezing cells of the pancreatic lineage, in particular endocrine progenitor cells. It also provides new pancreatic lineage cell populations.

### BACKGROUND

Induced pluripotent stem cells (iPSCs) derived by reprogramming of adult differentiated cells and human embryonic stem cells are uniquely suited for cell therapy applications because they are pluripotent and self-renewable, and can be differentiated into therapeutically useful cell types. Owing to the large variety of cell types that can arise in differentiating pluripotent stem cell (PSC) cultures, it is essential especially for cell therapy applications to develop methods for efficient directed differentiation of PSCs into the desired cell type(s), with low to absent fractions of less preferred or unwanted cell types. To generate large amounts of clinical grade cell material for implantation into patients, GMP-compatible, scalable manufacturing processes have to be developed, during which cell differentiation is performed in a closed system, ideally in suspension culture. To enable such processes, it is advantageous to perform differentiation of therapeutically important cell types such as pancreatic cells, including islet cells for diabetes therapy, by initially aggregating PSCs into cell clusters of a certain size distribution in suspension, and to subsequently perform a directed differentiation of these clusters via a defined succession of intermediate stages using selected growth factors and small molecule compounds. Key intermediate stages during differentiation into pancreatic islet-like clusters include definitive endoderm, primitive gut tube, pancreatic progenitor stage, and endocrine induction or endocrine stage. Protocols that contain steps with cells kept in 2D culture, and/or requiring dissociation of cells and re-association into clusters during differentiation, and/or sorting of progenitors or other desired cell intermediate or end stages are difficult to control, show poor scalability and lower yields, and are associated with higher reagent cost. Therefore, processes occurring in 3D suspension culture at all times are highly preferable.

Besides the key cell type for diabetes cell therapy, insulin-producing beta cells (either mature/glucose responsive or immature/KCl responsive), desired cell types in islet-like clusters differentiated from PSCs can include ARX^{pos} alpha cells or alpha cell precursors, polyhormonal endocrine cells (which have been shown to acquire an alpha cell fate after implantation) or somatostatin producing cells. Unwanted cells include residual pluripotent cells, which can pose a safety risk, non-endodermal cells, non-pancreatic endodermal cells, pancreatic ductal cells, and/or pancreatic enteroendocrine-like (EC-like) cells (Anlauf et al., J Histochem Cytochem. 2003 August; 51(8):1027-40, Carlei et al., Ital J Gastroenterol. 1993 May; 25(4):171-3.) producing serotonin/5-hydroxytryptamine and expressing TPH1, SLC18A1, FEV, CBLN1 and LMX1A. It should be noted that beta cells in adult humans may under certain circumstances also produce serotonin; however, the cells retain insulin/C-peptide expression. Some markers expressed in EC-like cells such as FEV and SLC18A1 may be expressed transiently also during differentiation into beta cells. Protocols for differentiation of iPSCs into beta cells are known in the art, e.g. from Nostro et al. (Stem Cell Reports. 2015 Apr. 14; 4(4):591-604), Rezania et al. (Nat Biotechnol. 2014 November; 32(11):1121-33) and Veres et al. (Nature. 2019 May; 569(7756):368-373), but lead to undesirably large proportions of unwanted cells.

Cryopreservation uncouples manufacturing from implantation into patients. It can improve the distribution of cells e.g. to local manufacturing or production centers for cell maturation and subsequent distribution to sites of clinical use. An efficient, GMP-compatible cryopreservation protocol is therefore a key element for cell therapy product development programs. Introducing controlled nucleation of ice formation in freezing protocols can overcome reproducibility and scalability issues associated with nucleation protocols based on induction by hand. Cell batches manufactured for therapeutic purposes in humans have to be tested in a range of assays before they can be released for therapeutic use, which may need several weeks to be completed and reported, meaning that on-demand-production of live cell therapy products from iPSCs is challenging. Cryopreservation provides time to conduct all required tests.

High recovery after cryopreservation means substantially lower overall cost for a cell therapy product. Differentiation of pluripotent stem cells uses many reagents and growth factors, all of which need to be available in GMP grade. Manufacturing under GMP itself is also highly cost intensive. Losing significant fractions of manufactured GMP batches during cryopreservation therefore has a substantial impact on overall cost of goods, and thereby eventually on treatment cost for the patient. Good overall recovery also likely means that surviving cells are in better functional state compared to low-recovery approaches, and that there is no need mitigate the effects of an excess of dead cells before administration.

Since the beginnings of human islet isolation research, attempts have been made to cryopreserve isolated human pancreatic islets (Kojayan et al., Islets (2018), 10(1): 40-49). However, while proof-of-concept in vitro results have been obtained, current clinical human islet transplantations invariably use fresh material (Bellin et al., Diabetologia (2020), 63(19): 2049-2056; Rickels and Robertson, Endocr Rev (2019), 40(2): 631-668).

Human islets and pluripotent stem cell-derived endocrine cell cluster material differ with regard to several parameters, for example cell composition, islet/cluster size, beta cell maturation state and stress sensitivity, making it unlikely that methods developed for human islet freezing are directly transferable to human pluripotent stem cell derived islet-like clusters.

For differentiation protocols yielding intermediate stages and end stage product as cell clusters in suspension, cryopreservation of intermediates or end stage products can be done either with intact cell clusters, or after dissociation of clusters into single cells.

Cryopreservation methods involving the dissociation and re-aggregation of pancreatic endocrine-stage clusters obtained in differentiation processes have been described, but it must be ensured that there are as few unwanted cells, such as EC-like cells and ductal cells, as possible.

Furthermore, dissociation of clusters into single cells requires the application of dissociation reagents such as proteases (e.g. trypsin), which, besides disrupting cell-cell contacts, may also digest other cell surface proteins and thereby have a negative impact on treated cells. Moreover, these reagents add cost, and need to be removed quantitatively from the final product to avoid adverse consequences upon product administration. Additionally, the dissociation of clusters is a laborious process, is difficult to control especially at larger scale, and holds a potential risk for contaminations. Typically, agents like ROCK inhibitors are added during re-aggregation, which add cost and are another component for which residual levels have to be monitored in the final product. Reproducible large-scale re-aggregation represents an additional technical challenge. Dissociation and re-aggregation is also associated with substantial loss of cell material, resulting in significantly reduced process cost effectiveness. Furthermore, the enrichment of serotonin-expressing EC-like cells by the dissociation and subsequent re-aggregation requires further measures to reduce the appearance of this population.

EP3521418 uses whole cluster freezing with cell populations containing 30% or more monohormonal beta cells, yet provides no information about cell recovery and viability post-thaw and post-recovery. EP3521418 states that methods described in U.S. Pat. No. 8,278,106 for pancreatic progenitors were used, except that CMRL-based media were used for post-thaw incubations. While U.S. Pat. No. 8,278,106 states that “at least 52% survival” was obtained when freezing progenitor cells, there is no quantitative information for freezing of endocrine cell containing-clusters in EP3521418. In vivo function of cryopreserved whole clusters is shown in EP3521418, but no details on post thaw recovery and viability are provided.

Single cell freezing of pancreatic endocrine-stage cells derived from human pluripotent stem cells is described in EP3681992. The re-aggregation step also associated with single cell freezing has been shown to enrich beta cells. However, the inventors observed that this approach enriches unwanted cells like serotonin-producing enteroendocrine-like cells.

These and further disadvantages need to be overcome. The present invention therefore addresses these needs and technical objectives and provides a solution as described in the following.

### SUMMARY

In a first aspect, the invention relates to a method of producing an islet-like cluster, comprising the steps of
i) providing an endocrine progenitor cell cluster,
ii) differentiating the endocrine progenitor cell cluster to an islet-like cluster comprising an incubation step iia) of incubating the endocrine progenitor cell cluster in the presence of a gamma secretase inhibitor and the absence of thyroid hormone, and an incubation step iib) of incubating the cell cluster resulting from step iia) in the absence of a gamma secretase inhibitor and of thyroid hormone.

In a second aspect, the invention relates to a cell culture comprising a plurality of islet-like clusters, characterized in that 25% or less of the cells in the cell culture are C-peptide^{neg} and serotonin^{pos}.

In a third aspect, the invention relates to a cell culture comprising a plurality of definitive endoderm cell clusters, characterized in that at least 85% of the cells in the cell culture are CXCR4^{pos} and EPCA^{pos}.

In a fourth aspect, the invention relates to a cell medium for differentiating an endocrine progenitor cell cluster towards an islet-like cluster, comprising a gamma secretase inhibitor and not comprising thyroid hormone.

In a fifth aspect, the invention relates to a method of producing a cryoprotected pancreatic lineage cell cluster, comprising the steps of
(i) providing a pancreatic lineage cell cluster in a medium,
(ii) cryoprotecting the pancreatic lineage cell cluster by cooling the medium to a temperature of about 8° C. to about −6° C. in the presence of increasing concentrations of ethylene glycol (EG) and increasing concentrations of dimethylsulfoxide (DMSO), wherein the final concentration of EG is at least about 1% v/v and the final concentration of DMSO is at least about 1% v/v, and
(iii) optionally freezing the cryoprotected pancreatic lineage cell cluster to a temperature of at least about −40° C., thereby producing a frozen cryoprotected pancreatic lineage cell cluster.

In a sixth aspect, the invention relates to a cryoprotective medium comprising a pancreatic lineage cell cluster, wherein the cryoprotective medium comprises at least about 1% v/v EG and at least about 1% v/v DMSO.

In a seventh aspect, the invention relates to a frozen cell culture comprising a plurality of pancreatic lineage cell clusters, characterized in that at least 20% of the cells are viable.

In an eighth aspect, the invention relates to a method of producing a thawed pancreatic lineage cell cluster, comprising the steps of
(i) providing a frozen pancreatic lineage cell cluster, and
(ii) thawing the frozen cell cluster.

In a ninth aspect, the invention relates to a thawed cell culture comprising a plurality of pancreatic lineage cell clusters, wherein the thawed cell culture is obtained from a culture of frozen cells and the recovery of viable cells is at least 20% of the frozen cells.

In a tenth aspect, the invention relates to the cells of cell culture of the second aspect for use as a medicament.

### LEGENDS TO THE FIGURES

FIG. 1 is a schematic illustration of the stages of differentiation of human pluripotent stem cells to islet-like clusters and the stages at which the new protocol elements (−T3=−Triiodothyronine, +GSI=+Gamma Secretase Inhibitor) are introduced (hPSC=human Pluripotent Stem Cell, DE=Definitive Endoderm, GT=Gut Tube, PP=Pancreatic Progenitor, EN=Endocrine Progenitor, ILC=Islet-Like Cluster containing NKX6.1/Insulin expressing beta cells and other cell types).

FIG. 2A shows representative flow cytometry plots of TLC cells obtained with the combination protocol (Control) or with the combination protocol with addition of GSI and removal of T3 (+GSI/−T3). Cells were stained for NKX6.1 and Chromogranin A (CHGA). Addition of GSI and removal of T3 from the EN stage onwards results in a highly enriched endocrine population.

FIG. 2B is a bar graph representation of flow cytometry analysis showing the significant reduction in the percentage of NK6.1^{pos}/CHGA^{neg} cells obtained with addition of GSI and removal of T3 (+GSI/−T3) in comparison to the combination protocol (Ctrl). (n=3, data are represented as mean±SD, *P<0.05).

FIG. 2C is a bar graph representation of flow cytometry analysis showing the significant increase of endocrine cells, obtained with addition of GSI and removal of T3 (+GSI/−T3) in comparison to the combination protocol (Ctrl), measured by CHGA expressing cells. (n=3, data are represented as mean±SD, *P<0.05).

FIG. 3A shows representative flow cytometry plots of ILC cells obtained with the combination protocol (Control) or with the combination protocol with addition of GSI and removal of T3 (+GSI/−T3). Cells were stained for NKX6.1 and C-peptide (C-PEPT).

FIG. 3B is a bar graph representing the percentage of NK6.1^{pos}/C-PEPT^{pos} cells obtained with the combination protocol (Ctrl) or with the combination protocol with addition of GSI and removal of T3 (+GSI/−T3) (n=3, data are represented as mean±SD, *P<0.05).

FIG. 4A shows representative flow cytometry plots of ILC cells obtained with the combination protocol (Control) or with the combination protocol with addition of GSI and removal of T3 (+GSI/−T3) and stained for Glucagon (GCG) and C-peptide (C-PEPT). Addition of GSI and removal of T3 from the EN stage onwards results in an increase of GCG^{pos} cells and thereby a cell composition more similar to human islets.

FIG. 4B is a bar graph representation of flow cytometry analysis showing the significant increase in the percentage of GCG^{pos} cells obtained addition of GSI and removal of T3 (+GSI/−T3) in comparison to the combination protocol (Ctrl). (n=3, data are represented as mean±SD, *P<0.05).

FIG. 5A shows representative flow cytometry plots of ILC cells obtained with the combination protocol (Control) or with the combination protocol with addition of GSI and removal of T3 (+GSI/−T3). Cells were stained for serotonin and C-peptide (C-PEPT). Addition of GSI and removal of T3 from the EN stage onwards results in reduction of the undesired serotonin^{pos}/C-peptide^{neg} population.

FIG. 5B is a bar graph representation of flow cytometry analysis showing the significant decrease in the percentage of serotonin^{pos}/C-peptide^{neg} cells obtained with addition of GSI and removal of T3 (+GSI/−T3) in comparison to the combination protocol (Ctrl). (n=3, data are represented as mean±SD, **P<0.01).

FIG. 6 shows representative flow cytometry plots of ILC cells obtained with the combination protocol (Control) or with the combination protocol with addition of GSI and removal of T3 (+GSI/−T3). Cells were stained for NKX6.1 and Islet1 (ISL1). The new conditions result in an increase of ISL1 expressing cells.

FIG. 7 is a panel of single cell RNA sequencing data represented as UMAP plots, comparing ILC cells obtained with the combination protocol (Control) to ILC cells obtained with the combination protocol with addition of GSI and absence of T3 (+GSI/−T3). In agreement with flow cytometry data, the endocrine population is enriched with the addition of GSI and absence of T3 (as seen with increased cell populations expressing INS, ISL1, ARX, SST, and the reduction of the SOX9 and CFTR-expressing cell population), and the serotonin-expressing population (as seen with TPH1 and SLC18A1 (VMAT1) expression) is reduced.

FIG. 8 shows representative flow cytometry plots of ILC cells obtained with the combination protocol (Control) or with the combination protocol with addition of GSI and removal of T3 (+GSI/−T3) and stained for NKX6.1 and Chromogranin A (CHGA). Addition of GSI and removal of T3 from the EN stage onwards results in a highly enriched endocrine population.

FIG. 9 shows representative flow cytometry plots of ILC cells obtained with the combination protocol (Control) or with the combination protocol with addition of GSI and removal of T3 (+GSI/−T3) and stained for NKX6.1 and VMAT1. Addition of GSI and removal of T3 from the EN stage onwards results in reduction of the VMAT1^{pos} population.

FIG. 10 shows representative flow cytometry plots of ILC cells obtained with the combination protocol (Control) or with the combination protocol with addition of GSI and removal of T3 (+GSI/−T3) and stained for NKX6.1 and Islet1 (ISL1).

FIG. 11 is a panel of single cell RNA sequencing data represented as UMAP (Uniform Manifold Approximation and Projection) plots, comparing ILC cells obtained with the combination protocol (Control) to ILC cells obtained with the combination protocol with addition of GSI and removal of T3 (+GSI/−T3). In agreement with flow cytometry data, the endocrine population is enriched with the addition of GSI and removal of T3 (as seen with increased cell populations expressing INS, ISL1, ARX, SST, and the reduction of the SOX9 and CFTR-expressing cell population), and the serotonin-expressing population (as seen with TPH1 and SLC18A1 expression) is reduced.

FIG. 12 shows the immunohistochemical staining of ILC clusters obtained with the combination protocol (Control) or with the combination protocol with addition of GSI and removal of T3 (+GSI/−T3). Histological sections were stained for C-peptide and serotonin. Nuclei were stained with DAPI. Bar=100 μm.

FIG. 13 shows the immunohistochemical staining of ILC clusters obtained with the combination protocol (Control) or with the combination protocol with addition of GSI and removal of T3 (+GSI/−T3). Histological sections were stained for C-peptide and SOX9. Nuclei were stained with DAPI. Bar=100 μm.

FIG. 14 is a panel of single cell RNA sequencing data represented as UMAP plots, comparing ILC cells obtained with the second differentiation protocol (Control) to ILC cells obtained with the second protocol with addition of GSI and removal of T3 (+GSI/−T3). In agreement with flow cytometry data, the endocrine population is enriched with the addition of GSI and removal of T3 (as seen with increased cell populations expressing INS, ISL1, ARX, SST, and the reduction of the SOX9 and CFTR-expressing cell population), and the serotonin-expressing population (as seen with TPH1 and SLC18A1 (VMAT1) expression) is reduced.

FIG. 15A is a bar graph quantifying the main pancreatic cell populations found in the scRNAseq analysis of FIG. 14.

FIG. 15B is a bar graph quantifying different undesired cell populations found in the scRNAseq analysis of FIG. 14.

FIG. 16 shows the immunohistochemical staining of ILCs obtained with the second protocol (Control) or with the second protocol with addition of GSI and removal of T3 (+GSI/−T3). Histological sections were stained for C-peptide and serotonin. Nuclei were stained with DAPI. Bar=100 μm.

FIG. 17 shows the immunohistochemical staining of ILCs obtained with the second protocol (Control) or with the second protocol with addition of GSI and removal of T3 (+GSI/−T3). Histological sections were stained for C-peptide and SOX9. Nuclei were stained with DAPI. Bar=100 μm.

FIG. 18 represents the blood glucose monitoring of diabetic mice implanted with ILCs obtained with the second protocol (Ctrl; n=4) or with ILC cells obtained with the second protocol with addition of GSI and removal of T3 (+GSI/−T3; n=6). 1.4 million beta cells/3 million total cells were transplanted under the kidney capsule of streptozotocin-treated NOD Scid Gamma mice. ILC cells generated with the second protocol with addition of GSI and removal of T3 are able to normalize blood glucose faster than ILC cells generated with the control condition, and maintain glycaemia at the human set point.

FIG. 19 shows representative flow cytometry panels of ILC cells obtained with the second protocol with addition of GSI and removal of T3.

FIG. 20 shows a representative example of an optimized cell composition in islet-like clusters obtained with the second protocol with addition of GSI and removal of T3, with levels of NKX6.1^{pos}/C-peptide^{pos} cells above 65% and only a small fraction of serotonin^{pos}/C-peptide^{neg} cells.

FIG. 21 shows representative flow cytometry plots of definitive endoderm induced with the combination protocol (Control) or with the combination protocol with addition of BMP4 and bFGF (+BMP4/+bFGF). The purity of the definitive endoderm, showed by high expression levels of CXCR4 and EPCAM, is higher with the addition of BMP4 and bFGF.

FIG. 22 shows a representative example of optimal cell composition of CXCR4hi/EPCAMhi expressing cells in definitive endoderm induced with the combination protocol with addition of BMP4 and bFGF.

FIG. 23 shows a schematic overview of islet-like cluster differentiation, cryopreservation and maturation time lines and an example of the ambient and sample temperature profile for cryopreservation of islet-like clusters using a programmed rate freezer.

FIG. 24a shows the recovery rates of post-thaw islet-like clusters obtained with the combination protocol (Ctrl) or with the combination protocol with addition of GSI and removal of T3 treatment after EN (+GSI/−T3). Recovery rates were calculated as follows: viable cell count of thawed islet-like clusters divided by the viable cell count before cryopreservation.

FIG. 24b is a bar graph representation of flow cytometry analysis of post-thaw islet-like cells obtained with the combination protocol (Ctrl) or with the combination protocol with addition of GSI and removal of T3 after EN (+GSI/−T3). Cells were stained for NKX6.1, C-peptide and Chromogranin A. Flow cytometry plots were pre-gated on viable single cells. Cell populations are shown relative to the respective cell population of the control.

FIG. 24c is a bar graph representation of flow cytometry analysis of post-thaw islet-like cells obtained with the combination protocol (Ctrl) or with the combination protocol with addition of GSI and removal of T3 after EN (+GSI/−T3). Cells were stained for C-peptide and serotonin. Flow cytometry plots were pre-gated on viable single cells. Cell populations are shown relative to the respective cell population of the control.

FIG. 24d is a bar graph representation of flow cytometry analysis of non-cryopreserved and post-thaw islet-like cells obtained with the combination protocol (Ctrl) or with the combination protocol with addition of GSI and removal of T3 after EN (+GSI/−T3). Cells were stained for C-peptide and serotonin. Flow cytometry plots were pre-gated on viable single cells. Differentiation of islet-like clusters in the presence of a GSI and absence of T3 improves the cell composition in both frozen/thawed conditions and non-cryopreserved conditions.

FIG. 25 shows the recovery rate of post-thaw islet-like clusters. Clusters, obtained with the second protocol, were cryoprotected and frozen at EN stage, EN stage+1 day (EN+1) and EN stage+2 days (EN+2) using cryoprotective medium containing 7.2% DMSO and 2.8% EG, thawed, and subsequently differentiated to islet-like clusters according to the second protocol with addition of GSI and removal of T3. Recovery rates are calculated as cell count of viable post-thaw islet-like cells divided by number of viable cells before cryopreservation.

FIG. 26a shows a panel of single cell RNA sequencing data represented as UMAP plots, comparing post-thaw islet-like clusters derived from the second differentiation protocol (Ctrl) or with the second protocol with addition of GSI and absence of T3 treatment (+GSI/−T3). The endocrine population is enriched with extended GSI and omission of T3 (as seen with increased cell populations expressing INS, ISL-1, ARX, SST, and the reduction of the SOX9 and CFTR-expressing cell population), and the serotonin-expressing population (as seen with TPH1, SLC18A1 (VMAT1), LMX1A and CBLN1 expression) is reduced.

FIG. 26b shows a bar graph quantifying the different cell populations highlighted in FIG. 26a.

FIG. 27a shows representative flow cytometry plots of islet-like clusters, obtained with the second protocol, stained for NKX6.1 and C-peptide (C-PEPT). Flow cytometry plots were pre-gated on viable single cells. The fraction of NKX6.1^{pos}/C-peptide^{pos} cells (beta-cells) in post-thaw islet-like clusters is comparable to the proportion in non-cryopreserved islet-like clusters.

FIG. 27b shows representative flow cytometry plots of islet-like clusters, obtained with the second protocol, stained for NKX6.1 and Chromogranin A (CHGA). Flow cytometry plots were pre-gated on viable single cells. Similar percentage of endocrine cells (CHGA^{pos}) between post-thaw islet-like clusters and non-cryopreserved islet-like clusters.

FIG. 27c shows representative flow cytometry plots of islet-like clusters, obtained with the second protocol, stained for C-peptide (C-PEPT) and serotonin, demonstrating that cryopreservation has no adverse effects on cell composition. Flow cytometry plots were pre-gated on viable single cells. Similar C-peptide and serotonin populations observed in post-thaw islet-like clusters and non-cryopreserved islet-like clusters.

FIG. 28a shows the recovery rate of post-thaw islet-like clusters derived from iPSCs, differentiated with the second protocol. EN+1 clusters were cryoprotected using medium containing varying amounts of DMSO and EG (as indicated in the figure) and subsequently frozen. Recovery rates are calculated as cell count of viable post-thaw islet-like cells divided by the number of viable cells before cryopreservation.

FIG. 28b shows the live/dead staining of thawed EN+1 clusters after the thawing process. Clusters were differentiated using the second protocol and frozen at EN+1 stage utilizing 4% DMSO and 6% EG and freezing procedure A. PI represents dead cells, FDA represents viable cells.

FIG. 28c shows the recovery rate of post-thaw islet-like clusters. Clusters, differentiated with the second protocol, were cryopreserved at EN+1 utilizing the cryoprotective medium A, cryoprotection treatment, and freezing procedure A in comparison to freezing medium and freezing procedure taken from EP3521418. FIG. 28c depicts data from two independent experiments. Recovery rates are calculated as cell count of viable post-thaw islet-like cells divided by the number of viable cells before cryopreservation.

FIG. 29a shows the recovery rate of post-thaw islet-like clusters. Clusters were differentiated using the second protocol, with extension of GSI in the absence of T3, and frozen at EN stage utilizing a cryoprotective medium containing 7.2% DMSO and 2.8% EG. After stepwise cryoprotection, the EN clusters were frozen with freezing procedure A in comparison to a freezing protocol including controlled induction of nucleation by means of an ambient temperature dip to −33° C. Recovery rates were calculated as followed: viable cell count of islet-like clusters divided by the viable cell count before cryopreservation.

FIG. 29b shows representative flow cytometry plots of post-thaw islet-like cells obtained with the second protocol with extended GSI and without T3 treatment and a freezing protocol including nucleation induction by an ambient temperature dip to −33° C., and stained for NKX6.1 and C-PEPT; NKX6.1 and ISL-1 or C-PEPT and serotonin after post-thaw differentiation. FACS plots were pre-gated on viable single cells.

FIG. 30 shows the recovery rate and live/dead staining of post-thaw clusters. Clusters were differentiated using the combination protocol and frozen at EN stage utilizing a cryoprotective medium containing the indicated percentages of DMSO and EG. A freezer protocol with an ambient temperature dip to −33° C. was applied. PI represents dead cells, FDA represents viable cells. Recovery rates were calculated as followed: viable cell count of islet-like clusters divided by the viable cell count before cryopreservation.

### DETAILED DESCRIPTION

Before the invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kolbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturers' specifications, instructions etc.), whether supra or infra, is hereby incorporated by reference in its entirety.

In the following, the elements of the invention will be described. These elements are listed with specific embodiments, however, it should be understood that they may be combined in any manner and in any number to create additional embodiments. The variously described examples and preferred embodiments should not be construed to limit the invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise. A preferred embodiment of the conjunction “and/or” whenever used herein is “and”.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

In a first aspect, the invention relates to method of producing an islet-like cluster, comprising the steps of
i) providing an endocrine progenitor cell cluster,
ii) differentiating the endocrine progenitor cell cluster to an islet-like cluster comprising an incubation step iia) of incubating the endocrine progenitor cell cluster in the presence of a gamma secretase inhibitor and the absence of thyroid hormone, and an incubation step iib) of incubating the cell cluster resulting from step iia) in the absence of a gamma secretase inhibitor and of thyroid hormone.

It is to be understood that during in vitro differentiation into pancreatic islet cells, transitions between cell stages may occur asynchronously, so a cell cluster may contain cells before (“progenitor cell type”), during (type of cell the cluster is named after) and after a differentiation stage (“successor cell type”). For example, at the endocrine progenitor (EN) stage (about five days after induction of endocrine differentiation in pancreatic progenitor cells), about 50-80% of the cells have successfully acquired an endocrine fate (CHGA^{pos}), and about 10-30% have progressed further to beta cell identity (CHGA^{pos}, NKX6.1^{pos} and insulin/C-peptide^{pos}). The remainder of cells still retain a progenitor phenotype or display transient NEUROG3 expression.

Thus, the term “cell cluster” with regard to a specific cell type (such as a definitive endoderm cell cluster, a gut tube cell cluster, a pancreatic progenitor cell cluster or an endocrine progenitor cell cluster, this being the order of differentiation) refers to an aggregation of cells of which substantially all (i.e. at least 70%, preferably at least 80%, more preferably at least 90% and most preferably at least 95%) are one of: the cell type the cluster is named after, the corresponding progenitor or successor cell type, or are at a transient stage between these types. Usually, the majority of the cells in the cell cluster are cells of the type the cell cluster is named after. Generally, the earlier the cell stage in the lineage, the more homogenous the cell cluster is with regard to the cell type it is named after. Accordingly, it is preferred that a definitive endoderm cell cluster comprises at least 90% (preferably at least 95%) definitive endoderm cells, a gut tube cell cluster comprises at least 85% (preferably at least 90% or 95%) gut tube cells, a pancreatic progenitor cell cluster comprises at least 70% (preferably at least 75%, 80%, 85%, 90% or 95%) pancreatic progenitor cells, and an endocrine progenitor (EN) cell cluster comprises at least 50% (preferably at least 70%, 80%, 85%, 90% or 95%) CHGA^{pos} endocrine progenitor cells. Preferably, the cells of a cluster are aggregated by the interaction of adhesion molecules expressed by the cells, such as cadherins. The shape of the cell cluster is preferably spheroid. With respect to size, the cell cluster preferably comprises about 50 to about 12,000 cells and/or has an average diameter of about 30 μm to about 600 μm. Cell clusters (as well islet-like clusters) described herein are suspension clusters, i.e. they are produced and kept in suspension culture. Preferably, all cells are human cells.

An “endocrine progenitor cell” is a cell of the pancreatic cell (or islet cell, specifically beta cell) lineage and represents a stage between pancreatic progenitor cell and islet cell (including beta cell). It can be characterized by expression of NKX6.1, NKX2.2, PDX1, NEUROD1 and CHGA. In the context of pluripotent stem cell to islet cell differentiation, the term “endocrine progenitor cell” also comprises CHGA^{pos}/NKX6.1^{neg}/PDX^{pos} cells that are capable of giving rise to dual- and polyhormonal endocrine cells, which upon implantation into a mammal and in vivo maturation are capable of giving rise to mostly glucagon-expressing alpha-like or alpha cells, as well as cells showing transient expression of NEUROG3 and SNAI2.

An “islet-like cluster” is a cell cluster which closely resembles naturally-occurring pancreatic islet cell structures at least in (i) size, (ii) morphology and/or (iii) types of hormones it is capable of producing (preferably (ii), more preferably (ii) and (iii), and most preferably (i), (ii) and (iii)). (i), (ii) and (iii) preferably mean:
(i) An average diameter from about 50 to about 400 μm, a volume from about 6×10⁴ μm³ to about 3.4×10⁷ μm³, and/or a number from about 200 to about 7,500 cells; preferably an average diameter from about 50 to about 300 μm, a volume from about 6×10⁴ μm³ to about 1.4×10⁷ μm³ and/or a number from about 200 to about 4,500 cells.
(ii) Comprising pancreatic beta cells, alpha cells, alpha cell precursors, dual- or polyhormonal cells which are capable of converting to monohormonal cells (typically alpha cells) post implantation (i.e. within the human organism), delta cells, epsilon cells, and pancreatic polypeptide cells (PP cells, also known as gamma cells or F cells).
(iii) Capable of producing, in particular secreting insulin, glucagon, pancreatic polypeptide, somatostatin and ghrelin.

An islet-like cluster may also contain fractions of ductal cells, pancreatic progenitors, or EC-like cells, as e.g. found in the ductal epithelium of adult primates. Because these cells have no anti-diabetic effects (EC-like cells, ductal cells), can differentiate into suboptimal cell compositions (pancreatic progenitors) and/or proliferate post implantation, minimizing these populations is a key objective of process optimization for pluripotent stem cell derived islet-like products. For example, the content of EC-like cells should be less than 20% or preferably less than 10%.

The cells of the islet-like cluster are aggregated by the interaction of adhesion molecules expressed by the cells, such as cadherins. The shape of the islet-like cluster is preferably spheroid, in particular if obtained directly with the method of the first aspect. However, other shapes are possible, such as sheets, strings or others. This is possible by reshaping, i.e. dissociating the cells of islet-like cluster directly obtained with the method of the first aspect and re-aggregating the cells in other shapes. Alternatively, clusters may also be aggregated without prior dissociation to larger structures. New shapes can be formed using random processes or directed processes, e.g. by using suitable molds and templates. Therefore, size feature (i) above applies in particular if the shape is spheroid and the cluster is obtained directly with the method of the first aspect, but not necessarily to reshaped clusters.

The species of the cells is preferably human. The islet-like clusters obtainable with the method of the first aspect are described in more detail with regard to the second aspect of the invention below.

“Beta cells” or “β cells” are pancreatic cells which express insulin, the pre-pro-insulin derived processing product C-peptide and NKX6.1. Endogenous (i.e. non-recombinant) insulin expression is linked to C-peptide expression in beta cells, i.e. so when it is referred to one herein, the other is included, too. Usually, C-peptide expression is assayed to determine endogenous insulin expression. Once beta cells have achieved expression of insulin/C-peptide and NKX6.1, they are fully specified, meaning that their fate is locked in irreversibly, and that they cannot give rise to other islet or pancreatic cell types such as alpha or ductal cells. Thus, a beta cell as referred to herein expresses at least insulin/C-peptide and NKX6.1. Acquisition of beta cell functionality and maturation is a stepwise process. Expression of NKX6.1 and C-peptide is maintained during this process. Important steps are: immature or fetal-like (basal insulin secretion but no response to glucose stimulation, plus a response, i.e. insulin secretion, to artificial membrane depolarization e.g. by KCl), mature and child-like (basal and glucose stimulated insulin secretion; response to artificial membrane depolarization), as well as mature and adult-like (basal and glucose stimulated insulin secretion; response to artificial membrane depolarization of the same or a lower magnitude than for glucose stimulation; and expression of SIX2 and SIX3). Accordingly, a beta cell in the islet-like cluster obtainable by the method of the first aspect may be an immature or a mature beta cell, e.g. child-like or adult-like. An immature beta cell can be characterized by the expression of C-peptide, NKX6.1, NKX2.2, PAX6, RFX3, PDX1, ISL1, MAFB, GLIS3 and MNX1. A mature beta cell can be characterized by the expression of C-peptide, NKX6.1, UCN3, MAFA, MAFB, IAPP, PDX1, RFX6, GLUT2, ISL1 and MNX1 (adult-like: further SIX2 and SIX3). With regard to maturity, step ii) of the method produces an islet-like cluster comprising immature beta cells, and may as such be termed an overall “immature islet-like cluster”. However, the method may comprise a step iii) of maturing the islet-like cluster of step ii) such that it comprises mature beta cells (“mature islet-like cluster”, including a “mature child-like islet-like cluster” and a “mature adult-like islet-like cluster” when comprising the respective mature beta cells). Means for maturing beta cells in vitro until they achieve child-like or adult-like maturation are well known in the art. Examples include an extended incubation in suitable maturation media, or extended incubation using a variety of stimuli and triggers. Examples are variations in glucose concentrations and circadian entrainment, treatment with BMP4, treatment with an Aurora kinase inhibitor and/or absence of Alk5 inhibitors. A preferred embodiment is the treatment of the islet-like clusters with an Aurora kinase inhibitor. In another embodiment, however, maturation is performed without treatment of the islet-like clusters with an Aurora kinase inhibitor. Furthermore, immature cells can be implanted into a mammal (e.g. a rodent or a human) to complete full maturation in situ. It is to be understood that when it is referred to beta cell maturation herein, the maturation of the islet-like cluster is meant, i.e. the beta cells are not matured separately from the cluster. Maturation of the islet-like cluster does not necessarily mean that other cells in the cluster are also matured.

In one embodiment, the method does not comprise an in vitro maturation step, but produces an immature islet-like cluster for implantation as described above. Manufacturing and implanting islet-like clusters containing immature beta cells into patients may be preferred, as a) a shorter manufacturing time is needed to produce them (and hence manufacturing costs are lower) compared to mature beta cell containing clusters, which need additional time for maturation, and b) the inventors anticipate that such clusters will be more resilient against post-implantation stress e.g. due to transient initial hypoxia.

Thyroid hormone (triiodothyronine, T3) has been linked to pancreatic cell differentiation and maturation, and thyroid hormone has been used to promote beta cell differentiation and maturation in protocols for the differentiation of human pluripotent stem cells into insulin-secreting beta cells (e.g. Aguayo-Mazzucato et al., J Clin Endocrinol Metab. 2015 October; 100(10):3651-9.). The main mechanism of action by which thyroid hormone exerts its effects on human cells is by binding to thyroid hormone receptors (THRs) alpha and/or beta, members of the nuclear hormone receptor class of transcriptional regulators. The inventors found that, unexpectedly, the differentiation of endocrine progenitor cell clusters is improved when these cells are differentiated without thyroid hormone, but with a gamma secretase inhibitor in a first incubation step, and that differentiation is further improved by removing the gamma secretase inhibitor before differentiation to islet-like clusters is concluded.

While other steps may be comprised in step ii), it is to be understood that thyroid hormone is absent during the entire step ii). It is also to be understood that the cell clusters are not incubated in step ii) in the presence of a functional equivalent of thyroid hormone, as this would be technically nonsensical in view of the definition of step ii). Thus, it is preferred that not only thyroid hormone, but also precursors thereof, as well as other thyroid hormone receptor agonists are absent in step ii). For example, the precursor thyroxine (T4), which is enzymatically converted to thyroid hormone in human cells, is also absent from step ii). Since THRs can also be activated by structurally unrelated synthetic agonists, such as GC-1, at least GC-1, but preferably any thyroid hormone receptor agonist is also absent from step ii). Alternatively, step ii) can also be defined with respect to thyroid hormone and functional equivalents as differentiating the endocrine progenitor cell cluster to an islet-like cluster without activating THR alpha or beta in the cell cluster.

The gamma secretase inhibitor is, in a preferred embodiment, selected from the group consisting of gamma secretase inhibitor XXI (CAS 209986-17-4), XX (CAS 209984-56-5), IX (CAS number 208255-80-5), LY411575 (CAS number 209984-57-6), LY3039478 (CAS 1421438-81-4), DBZ (CAS 209984-56-5), LY450139 (CAS 425386-60-3), L-685,458 (CAS 292632-98-5), DAPT (CAS 208255-80-5), PF 3084014 (CAS 1962925-29-6), PF06648671 (CAS 1587727-31-8), Avagacestat (CAS 1146699-66-2), BMS299897 (CAS 290315-45-6), BMS906024 (CAS 1401066-79-2), R04929097 (CAS 847925-91-1), ibuprofen (CAS 51146-56-6), fenofibrate (CAS 49562-28-9), sulindac (CAS 38194-50-2), flurbiprofen (CAS 5104-49-4), indomethacin (CAS 53-86-1), GSM-1 (CAS 884600-68-4), E-2012 (CAS 870843-42-8), MK-0752 (CAS 471905-41-6), MRK-560 (CAS 677772-84-8), Itanapraced (CAS 749269-83-8), BPN-15606 (CAS 1914989-49-3), and Fosciclopirox (CAS 1380539-06-9). Preferably, it is gamma secretase inhibitor XXI or LY411575. Functionally equivalent compounds exist and can be used instead.

It is to be understood that if step iia) is carried out in the presence of a particular gamma secretase inhibitor, step iib) is carried out not only in the absence of this particular gamma secretase inhibitor, but also in the absence of the other gamma secretase inhibitors exemplified above, in particular in the absence of any gamma secretase inhibitor, as replacing the gamma secretase inhibitor of step iia) with a functional equivalent would be technically nonsensical in view of the definition of step ii). Furthermore, it is to be understood that step ii) does not comprise a further step subsequent to steps iia) or iib) in which a gamma secretase inhibitor is present. Alternatively, step ii) can also be defined with respect to gamma secretase inhibitors as differentiating the endocrine progenitor cell cluster to an islet-like cluster comprising inhibiting gamma secretase in step iia) and not comprising inhibiting gamma secretase in step iib) (or any step subsequent to step iia)).

In a preferred embodiment, step ii) consists of steps iia) and iib).

“In the absence” of a factor may, in a preferred embodiment, mean that the factor was comprised in the previous medium.

Regarding the duration of the incubation of step iia), it is preferred that the cell cluster is incubated for about 2 to about 6 days, preferably about 3 to about 5 days, more preferably about 4 days. “About” with respect to days herein preferably means±6 hours, more preferably ±4 hours, most preferably ±2 hours. Step iib) is preferably carried out until the endocrine progenitor cell cluster has been differentiated to an islet-like cluster. To achieve this, the cell cluster can be incubated for about 5 to about 9 days, preferably about 6 to about 8 days, more preferably about 7 days. It is preferred that the duration of step ii) in total is about 7 to about 15 days, preferably about 9 to about 13 days, more preferably about 11 days. The islet-like cluster may be matured as described above (e.g. into a child-like or adult-like mature islet-like cluster).

In a preferred embodiment, step iia) incubates the endocrine progenitor cell cluster in the presence of a suitable basal medium, an inhibitor of BMP signaling, an Alk5 inhibitor such as Alk5i II, and/or zinc; and/or step iib) incubates the cell cluster in the presence of a suitable basal medium, an inhibitor of BMP signaling, an Alk5 inhibitor such as Alk5i II, and/or zinc, and optionally a polyanionic polymer. The polyanionic polymer may be used throughout step iib) or only for a part of step iib), e.g. for at least about 1, 2, 3, 4 or 5 days. The use of an inhibitor of BMP signaling and an Alk5 inhibitor may also be defined as the step comprising inhibiting BMP signaling and comprising inhibiting Alk5, respectively.

Step i) comprises providing an endocrine progenitor cell cluster by any means and does not require producing it. In one embodiment, it is obtained by aggregating endocrine progenitor cells. In another embodiment, it is obtained by differentiation from prior stage cells of the pancreatic lineage, such as pancreatic progenitor cells or a cluster thereof, gut tube cells or a cluster thereof, or definitive endoderm cells or a cluster thereof. It may also be obtained by differentiation from pluripotent cells or a cluster thereof. In case it is obtained from cells of a certain stage rather than a cell cluster of cells of that stage, step i) comprises the aggregation to a cell cluster at any stage. In a preferred embodiment, step i) comprises
ia) providing a pluripotent cell cluster, e.g. by aggregating pluripotent cells,
ib) differentiating the pluripotent cell cluster to a definitive endoderm cell cluster,
ic) differentiating the definitive endoderm cell cluster to a gut tube cell cluster,
id) differentiating the gut tube cell cluster to a pancreatic progenitor cell cluster, and
ie) differentiating the pancreatic progenitor cell cluster to an endocrine progenitor cell cluster.

A “pluripotent cell” or “pluripotent stem cell” refers to a cell, preferably a human cell, that is capable of differentiation into any cell type of the respective organism. The pluripotent cell may be an embryonic stem cell (ESC), or it may be an induced pluripotent stem cell (iPSC). It can be characterized by the expression of a pluripotency marker such as OCT4, NANOG, LIN28A, ESRG, SOX2, SSEA4, TRA-1-60 and/or TRA-1-81. Many iPSC cell lines are available for use in context with the invention, such as iPSC6.2/GibcoEpi, iPS11, iPS15, F002.1A.13, hiPSC-1, hiPSC-2, LiPSC-GR1.1, LiPSC-GR1.2, HEL24.3, HEL113.5-corrected, CGT-RCiB-10, MHHi001-A, MHHi006-A, MHHi008-A, MHHi008-B, MHHi008-C, VC645-9, VC913-5, VC618-3, VC646-1, iPS 1016, or iPS 1031. Preferably, it is a clinical grade or GMP-grade iPSC line. It should be noted that iPSC lines can also be generated in a patient-specific manner for personalized/autologous applications, preferably as GMP-grade/clinical grade lines.

Similarly, many hESC lines, including also clinical/GMP-grade hESC lines are available for use in context with the invention, such as H1, H9, HUES8, CyT49, MEL1, KCLO37, RC-09, RC-11, 16, MAN10/11/12, MAN14/15/16, ESI-013, ESI-014, ESI-017, ESI-051, ESI-027, ESI-035, ESI-049, ESI-053, MasterShef2, MasterShef7 or MasterShef10. Preferably, it is a clinical grade or GMP-grade ESC line.

A “definitive endoderm cell” is a cell of the pancreatic cell lineage and represents a stage between a pluripotent cell and a gut tube cell. It can be characterized by the expression of SOX17, CXCR4, CD 117 and EPCAM.

A “gut tube cell” is a cell of the pancreatic cell lineage and represents a stage between a definitive endoderm cell and a pancreatic progenitor cell. It can be characterized by the expression of HNF1B and HNF4A.

A “pancreatic progenitor cell” is a cell of the pancreatic cell lineage and represents a stage between a gut tube cell and to an endocrine progenitor cell. It can be characterized by the expression of NKX6.1 and PDX1, and preferably also PTF1A, SOX9, C-MYC and CPA1.

Other cell types are defined above.

General procedures and steps which can be employed for the differentiation steps are generally known in the art (see, e.g., Rezania et al., Nature Biotech (2014), 32: 1121-1133 or Nostro et al., Stem Cell Reports (2015), 4(4): 591-604) and are also given in the examples herein.

In a particular embodiment, step ib) differentiates in the presence of a SMAD and MAPK signaling activator such as BMP4, a fibroblast growth factor (preferably one capable of binding to FGFR2 and FGFR3, such as FGF2, also known as bFGF, or FGF1) and a polyanionic polymer. The presence of the former two may also be defined as step i) comprising activating SMAD and MAPK signaling and comprising activating FGF receptor signaling, in particular signaling of FGFR-2 and optionally also FGFR-3. Step ib) can be characterized further by the use of a suitable basal medium, activating TGFβ signaling (i.e. by differentiating in the presence of a TGFβ signaling activator such as Activin A), activating WNT signaling (i.e. by differentiating in the presence of a WNT signaling activator such as CHIR99021, WNT3A or a GSK3 inhibitor) and/or inhibiting ROCK (i.e. by differentiating in the presence of a ROCK inhibitor such as Y-27632).

Optionally, after step ie), the endocrine progenitor cell cluster is cryoprotected and preferably frozen. In a preferred embodiment, this is done according to the methods of cryoprotecting and freezing described below.

In fact, it is generally possible that the endocrine progenitor cell cluster provided in step (i) is frozen and step (i) comprises thawing the frozen endocrine progenitor cell cluster, preferably thawing according to the methods described below. The inventors have found that the method of the first aspect is particularly advantageous for the differentiation of endocrine progenitor cells or clusters thereof that were frozen, in particular according to the methods described below.

Definitions and embodiments described below, in particular under the header ‘Definitions and further embodiments’ apply to the first aspect.

In a second aspect, the invention relates to a cell culture comprising a plurality of islet-like clusters, characterized in that 25% or less of the cells in the cell culture are C-peptide^{neg} and serotonin^{pos}. Preferably 20% or less, more preferably 15% or less, or even 8% or less of the cells in the cell culture are C-peptide^{neg} and serotonin^{pos}. This cell marker profile represents pancreatic enteroendocrine-like (EC-like) cells, which can arise during the differentiation process but which are undesired. In a preferred embodiment, the cell culture is further characterized in that at least 40%, preferably at least 45%, at least 50%, or even at least 55% of the cells in the cell culture are beta cells (preferably immature beta cells). To the inventors' best knowledge, an islet-like cluster culture with such a low level of EC-like cells, in particular combined with such a high percentage of (immature) beta cells has not been achieved before in differentiation processes. EC-like cells can further be characterized by expressing VMAT1 (SLC18A1), FEV, CBLN1 and LMX1A. The islet-like cluster can be matured further in vitro or in vivo.

Accordingly, it is preferred that the cell culture (i.e. the level of C-peptide^{neg} and serotonin^{pos} cells and preferably of (immature) beta cells) (i) is obtainable by differentiation of endocrine progenitor cell clusters to islet-like clusters, and/or (ii) has not been obtained by cell purification methods involving dissociation of the clustered cells, such as FACS or sorting with magnetic beads. In the most preferred embodiment, the islet-like clusters of the cell culture are obtainable by the method of the first aspect.

The cell culture can be further characterized as follows:
1) at least 40%, preferably at least 45%, more preferably at least 50% or even at least 55% of the cells are NKX6.1^{pos} and C-peptide^{pos}, wherein these cells preferably are immature beta cells,
2) less than 15%, preferably less than 10%, more preferably less than 5%, most preferably less than 3% or even 2% of the cells are ductal cells,
3) no detectable level of CDX2^{pos}/VMAT1^{pos} cells (preferably not detectable by flow cytometry detecting CDX2 and VMAT1),
4) 5% or less, preferably 2.5% or less of the cells are VMAT1^{pos} cells,
5) at least 30%, preferably at least 35% of the cells are NKX6.1^{pos} and ISL1^{pos} cells,
6) at least 30%, preferably at least 40%, more preferably at least 45% of the cells are ISL1^{pos} cells,
7) at least 80%, preferably at least 90%, more preferably at least 95% of the cells are CHGA^{pos} endocrine cells,
8) at least 5%, preferably at least of the cells are 10% ARX^{pos} cells,
9) at least 10%, preferably at least of the cells are 15% SST^{pos} cells,
10) at least 10%, preferably at least 15% of the cells are GCG^{pos} cells,
11) 2% or less, preferably 1% or less, more preferably 0.6% or less PDX1^{neg}/Ki67^{pos} cells; and/or 2% or less, preferably 1% or less, more preferably 0.5% or less or even 0.2% or less of the cells are NKX6.1^{neg}/Ki67^{pos} cells and/or,
12) less than 2.5% of the cells are SOX^{pos}/CHGA^{pos} endocrine cells.

The above order represents a ranking in preference. Preferred combinations include feature 1) and one or more of features 2) to 12) or preferably 2) to 8). Examples of preferred feature combinations are: 1)+2)+3)+8) or 10); 1)+2)+4)+8) or 10); 1)+2)+5) or 6)+11); 1)+2)+7)+8), 1)+2)+3)+7)+9); and 1)+2)+12).

It is to be understood that substantially all cells (e.g. at least 90%, 95% or 99%) of the culture are clustered cells as described above.

Definitions given and embodiments described with respect to the first aspect apply also to the second aspect, in as far as they are applicable. Also, definitions and embodiments described below, in particular under the header ‘Definitions and further embodiments’ apply to the second aspect.

In a third aspect, the invention relates to a cell culture comprising a plurality of definitive endoderm cell clusters, characterized in that at least 85% of the cells in the cell culture are CXCR4^{pos} and EPCAM^{pos}. Preferably at least 90%, more preferably at least 92%, most preferably at least 95%, 96% or even 97%, 98% or 99% of the cells are CXCR4^{pos} and EPCAM^{pos}. These cells can be characterized further in that the expression is high, i.e. the cells are CXCR4^{hi} and EPCAM^{hi}. Accordingly, in a preferred embodiment, CXCR4^{pos} herein may be replaced with CXCR4^{hi}, and EPCAM^{pos} herein may be replaced with EPCAM^{hi}. A “hi” co-expressing cell population can be understood as a cell population with a tight range of high expression intensity, i.e. an expression by all cells of the population that is clearly higher than in mesodermal cells and/or in neuroectodermal cells of the same developmental stage, or higher than in pluripotent cells (e.g. with at least a mean fluorescent intensity 3 fold higher than for other cell populations).

Furthermore, it is preferred that at least 85% of the cells in the cell culture are CXCR4^{pos}, EPCAM^{pos} and SOX17^{pos}; preferably at least 90%, more preferably at least 92%, most preferably at least 95%, 96% or even 97%, 98% or 99% of the cells are CXCR4^{pos}, EPCAM^{pos} and SOX17^{pos}.

It is preferred that the cell culture (i.e. the level of CXCR4^{pos}/EPCAM^{pos} cells) (i) is obtainable by differentiation of pluripotent cell clusters to definitive endoderm cell clusters, and/or (ii) has not been obtained by cell enrichment or purification methods such as FACS or sorting with magnetic beads. In the most preferred embodiment, the definitive endoderm cell clusters of the cell culture are obtainable by the method according to steps ia) and ib) of the method of the first aspect.

The third aspect also relates to method according to these steps and not necessarily including other steps of the method of the first aspect, i.e. to a method of producing a definitive endoderm cell cluster, comprising the steps of
i) providing a pluripotent cell cluster, e.g. by aggregating pluripotent cells,
ii) differentiating the pluripotent cell cluster to a definitive endoderm cell cluster in the presence of a SMAD and MAPK signaling activator, a fibroblast growth factor and a polyanionic polymer,
as described above for steps ia) and ib) of the method of the first aspect.

Preferably, the method produces a plurality of such clusters, and thereby a cell culture according to the third aspect.

The third aspect also relates to a medium for differentiating a pluripotent cell cluster to a definitive endoderm cell cluster, the medium comprising a SMAD and MAPK signaling activator, a fibroblast growth factor and a polyanionic polymer, as described above.

Definitions given and embodiments described with respect to the first and second aspect apply also to the third aspect, in as far as they are applicable. Also, definitions and embodiments described below, in particular under the header ‘Definitions and further embodiments’ apply to the third aspect.

In a fourth aspect, the invention relates to a cell medium for differentiating an endocrine progenitor cell cluster towards an islet-like cluster, comprising a gamma secretase inhibitor and not comprising thyroid hormone.

It is to be understood that the cell medium does not comprise a functional equivalent of thyroid hormone either, such as a precursor or another thyroid hormone receptor agonist, as explained with regard to the method of the first aspect.

It is preferred that the cell medium is a suitable basal cell medium supplemented with and thus comprising a gamma secretase inhibitor, which is preferably further supplemented to comprise an inhibitor of BMP signaling, an Alk5 inhibitor and/or zinc. Optionally, it may further be supplemented to comprise a polyanionic polymer.

Similarly, the invention relates to the use of the cell medium as defined for differentiating an endocrine progenitor cell cluster towards an islet-like cluster.

“Towards” means that the cell cluster achieved may be (e.g. if the polyanionic polymer is included), but does not have to be an islet-like cluster. In case of the latter, it is a cell cluster that can develop into an islet-like cluster.

The invention also relates to a kit comprising a cell medium of the fourth aspect. In a preferred embodiment, the kit comprises a first cell medium of the fourth aspect, characterized in that it does not comprise a polyanionic polymer, and a second cell medium of the fourth aspect, characterized in that it does comprise a polyanionic polymer. This kit is particularly useful for use in the method of the first aspect.

In a preferred embodiment, the kit further comprises a frozen cell culture according to the seventh aspect.

The kit may also comprise a cell medium comprising a SMAD and MAPK signaling activator, a fibroblast growth factor such as FGF2 and a polyanionic polymer. The kit comprising this medium is particularly useful for use in the method of the first aspect comprising step ib). It is preferred that the cell medium is a suitable basal cell medium supplemented with these components. This basal cell medium may further be supplemented to comprise a TGFβ signaling activator, a WNT signaling activator, and/or a ROCK inhibitor.

Exemplary/preferred embodiments of components of any of these cell media are indicated elsewhere herein.

Definitions given and embodiments described with respect to the first, second and third aspect apply also to the fourth aspect, in as far as they are applicable. Also, definitions and embodiments described below, in particular under the header ‘Definitions and further embodiments’ apply to the fourth aspect.

In a fifth aspect, the invention relates to a method of producing a cryoprotected pancreatic lineage cell cluster, comprising the steps of
(i) providing a pancreatic lineage cell cluster in a cell medium,
(ii) cryoprotecting the pancreatic lineage cell cluster by cooling the cell medium to a temperature of about 8° C. to about −6° C. in the presence of increasing concentrations of ethylene glycol (EG) and increasing concentrations of dimethylsulfoxide (DMSO), wherein the final concentration of EG is at least about 1% v/v and the final concentration of DMSO is at least about 1% v/v, and
(iii) optionally freezing the cryoprotected pancreatic lineage cell cluster to a temperature of at least about −40° C., thereby producing a frozen cryoprotected pancreatic lineage cell cluster.

The inventors found that the method of the fifth aspect results in superior recovery and viability of such cell clusters after thawing. The cryoprotected pancreatic lineage cell clusters may be kept frozen for any amount of time, e.g. at least about 1 hour, 6 hours, 1 day, 1 week, 1 year or multiple years or decades. Typically, such cryostorage will be for periods of time sufficiently long to allow quality control (QC) assays to be performed, and according to the requirements of clinical applications. However, the inventors have not detected low recovery due to long storage times and do not anticipate this in particular for low temperatures (e.g. −120° C. or lower), in agreement with the absence of metabolic, chemical or phase changes expected in cells stored at such low temperatures.

The volume of a cryoprotected (or likewise a frozen or thawed) cell culture herein may for example be from about 0.1 ml to about 10 ml, preferably from about 0.5 ml to about 2 ml, more preferably about 1 ml, in particular if a cryovial (typically of cylindrical shape) is used as a container. However, larger volumes may be used in suitable containers, such as cryobags (typically flat and flexible, allowing for rapid and homogenous freezing of large volumes), allowing for cell culture volumes of e.g. from about 5 ml to about 350 ml, preferably from about 10 ml to about 275 ml, more preferably from about 30 ml to 100 ml.

The cell medium may be described as a basal cryoprotective medium which is suitable, with the addition of EG and DMSO as described herein, for cryoprotecting and freezing the cells described herein. It preferably comprises amino acids (e.g. one or more (or all) of glycine, L-histidine, L-isoleucine, L-methionine, L-phenylalanine, L-proline, L-hydroxyproline, L-serine, L-threonine, L-tryptophan, L-tyrosine, and L-valine), trace elements (e.g. one or more (or all) of Ag, Al, Ba, Br, Cd, Co, Cr, F, Ge, I, Mn, Mo, Ni, Rb, Se, Si, Sn, V, and Zr), vitamins/antioxidants (e.g. one or more (or all) of thiamine, reduced glutathione, ascorbic acid, and 2-PO₄), and proteins (e.g. one or more (or all) of transferrin (preferably iron-saturated), insulin, albumin (preferably lipid-rich, such as AlbuMAX)). Examples are KO-SR medium (knockout serum replacement, e.g., KnockOut™ Serum Replacement, which is available from Thermo Fischer Scientific, MA, USA) or KnockOut™ Serum Replacement CTS Xeno-Free (Thermo Fisher Scientific), a composition containing the same components as KO-SR but replacing animal-derived components such as bovine albumin with recombinant human serum albumin (e.g. using Cellastim, Invitria), TCH® defined serum replacement (Fisher Scientific) or Serum Replacement 1 (Merck).

A pancreatic lineage cell cluster may generally be a cluster of any cell type of the pancreatic lineage, e.g. a definitive endoderm cell cluster, a gut tube cell cluster, a pancreatic progenitor cell cluster, an endocrine progenitor cell cluster or an islet-like cluster. In a preferred embodiment, wherever referred to herein with regard to cryoprotection, freezing or thawing, the pancreatic lineage cell cluster is a definitive endoderm cell cluster, a gut tube cell cluster, a pancreatic progenitor cell cluster or an endocrine progenitor cell cluster, preferably a gut tube cell cluster, a pancreatic progenitor cell cluster or an endocrine progenitor cell cluster, more preferably a pancreatic progenitor cell cluster or an endocrine progenitor cell cluster, most preferably an endocrine progenitor cell cluster. In this embodiment, it is preferred that the endocrine progenitor cell cluster provided in step (i) is within about 1 to about 3 days, preferably within about 1 to about 2 days, more preferably within about 1 day after reaching the endocrine (EN) stage. “Reaching” shall mean that at least 50% of the cells of the cluster are CHGA^{pos}. Preferably, it means day five after inducing the EN stage, wherein the time point of inducing is the contacting of the progenitor cell cluster with differentiation medium for differentiating pancreatic progenitor cells to endocrine progenitor cells. The inventors have shown that cryoprotecting such early endocrine progenitor cell clusters increases recovery (i.e. the number of living cells after freezing and thawing).

In step (ii), the cell medium is preferably cooled to a temperature of about 6° C. to about −2° C., more preferably of about 4° C. to about 0° C. The final concentration of EG preferably is from about 5% to about 9% v/v, more preferably from about 5% to about 8.5% v/v (even more preferably from about 5.5% to about 8% v/v), and most preferably from about 5.5% to about 6.5% v/v. The final concentration of DMSO preferably is from about 3% to about 9% v/v, more preferably from about 3% to about 8% v/v, and most preferably from about 3% to about 5% v/v. The final concentration of EG and DMSO combined preferably is from about 7% to about 13% v/v, more preferably from about 9% to about 12.5% v/v, and most preferably from about 9% to about 11% v/v.

“Increasing concentrations” means that during step (ii), the concentrations of EG and DMSO are increased, preferably stepwise (i.e. not continuously) and more preferably associated with decreasing temperature (e.g. in at least one of the steps the temperature is decreased compared to a previous step that involved an increase of EG and DMSO). In one embodiment, the final concentrations of EG and DMSO are obtained by steps comprising:
a) obtaining a first concentration of EG and DMSO and cooling the medium to a temperature from about 24° C. to about 18° C.;
b) obtaining a second concentration of EG and DMSO and cooling the medium to a temperature from about 8° C. to about −6° C.; and
c) obtaining a third concentration of EG and DMSO and maintaining the medium at the temperature of step b) or cooling the medium to a temperature from about 8° C. to about −6° C. that is lower than the temperature of step b).

Therein, a concentration can be obtained by adding EG and/or DMSO to the medium, or by replacing the medium with a medium comprising the prescribed concentrations of EG and/or DMSO (the transfer of cell clusters to another container with a different medium is, in the context of the invention, considered as replacing the medium). The concentration of EG and/or DMSO increases with each step. The first concentration of EG and/or DMSO is lower than the final concentration, which is described above, preferably by a factor ranging from about 2.2 to about 3.9, preferably from about 2.5 to about 3.6, more preferably from about 2.7 to about 3.5, most preferably from about 2.9 to about 3.2. The second concentration of EG and/or DMSO is lower than the final concentration preferably by a factor ranging from about 1.3 to about 2.4, preferably from about 1.5 to about 2.2, more preferably from about 1.7 to about 2.0, most preferably from about 1.8 to about 1.9. Therein, the second concentration is higher than the first concentration. The third concentration of EG and DMSO is higher than the second concentration and preferably is the final concentration. It is to be understood that further steps with intermediate concentrations can be performed prior to steps a), b) or c) (and also after step c) if the third concentration is not the final concentration). Such further steps may be at the temperature of the previous or subsequent step, or in between.

In each step, the medium is cooled by reducing the ambient temperature accordingly and maintaining the medium at this temperature for a duration of time. For example: in step a), the duration may be at least about two minutes, preferably about 5 minutes; in step b), the duration may be at least about 15 minutes, preferably for at least about 20 minutes, more preferably for about 25 minutes; in step c), the duration may be at least about 5 minutes, preferably for at least about 10 minutes, more preferably for about 15 minutes.

In a preferred embodiment, the cryoprotected pancreatic lineage cell cluster is frozen, preferably comprising the steps of:
1) cooling the medium to a temperature of about −5° C. to about −10° C. at a cooling rate avoiding ice nucleation,
2) incubating the medium at the temperature of step 1) until an even temperature distribution throughout the medium is achieved,
3) decreasing the ambient temperature to at least about −30° C. (e.g. to about −33° C.) to induce ice nucleation,
4) increasing the ambient temperature above the ambient temperature of step 3) and maintaining the temperature until ice has propagated throughout the medium, and
5) cooling the medium to a temperature of at least about −40° C.

“Cooling the medium to a temperature” means that at least in one region of the medium, the indicated temperature is reached.

The “cooling rate avoiding ice nucleation” (step 1)) can be determined by the skilled person without undue burden. In a preferred embodiment, it is up to about 2.5° C. per minute, preferably up to about 2.0° C. per minute, more preferably up to about 1.7° C. per minute and most preferably up to about 1.6° C. per minute, e.g. about 1.5° C. per minute. The temperature cooled to in step 1) preferably is about −6° C. to about −9° C., more preferably about −7° C. to about −8° C. and most preferably about −7.5° C.

The incubation time of step 2) depends on the temperature cooled to in step 1) and on the cooling rate, and it can be determined by the skilled person without undue burden. At the given temperatures and rates, it is at least about 3 minutes, preferably at least about 5 minutes, more preferably at least about 8 minutes and most preferably about 10 minutes. While an upper time limit is not critical, for convenience it is envisaged that it is no longer than about 25 minutes, preferably no longer than about 20 minutes, more preferably no longer than about 15 minutes.

In step 3), the ambient temperature is preferably decreased to at least about −40° C., more preferably to at least about −50° C., most preferably to at least about −60° C., e.g. to about −66° C. This decrease is usually sudden, preferably at a rate of at least about 50° C. per minute, more preferably at least about 70° C. per minute, most preferably at least about 90° C. per minute, e.g. about 99° C. per minute. This temperature dip induces ice nucleation.

Other ways of inducing ice nucleation may be used instead, however, such as inducing a supercool spot (e.g. lower than −50° C., e.g. of about −80° C.), are possible, usually using a suitable device. This may take place at the container wall or within the culture, and both can be achieved by using a device such as a cryopen (a device which, by expelling liquid nitrous oxide, can cool a small area to below −50° C.) or a pre-cooled tool such as forceps. Of the two, inducing a supercool spot at the outer container wall without opening the container is preferred. Nucleation can also be induced by mechanical agitation. Alternatively, although less preferred as it requires opening the container, nucleation can be achieved by adding an ice-nucleating agent to the medium. Generally, nucleation methods which avoid opening the container are preferred, thus ensuring sterility of this part of the process. In other words, ice nucleation is preferably induced without opening the container comprising the pancreatic lineage cell cluster.

Inducing nucleation, i.e. the formation of ice, during the cryopreservation process, to avoid supercooling and harming the cells by release of latent heat, is beneficial for cell survival. Introducing a temperature dip in the cryopreservation protocol to induce nucleation overcomes the bottlenecks for reproducibility, GMP-compatibility and upscaling of freezing protocols, and therefore is preferred relative to other methods described herein.

In step 4), the ambient temperature is increased to above about −60° C., preferably to above about −50° C., more preferably to above about −40° C., most preferably to above about −30° C., e.g. to about −28° C., or even to above about −20° C., −15° C. or −10° C. The upper temperature is such that ice still forms and can propagate throughout the medium. The increase is usually sudden, preferably at a rate of at least about 30° C. per minute, more preferably at least about 50° C. per minute, most preferably at least about 70° C. per minute, e.g. about 72° C. per minute. The inventors found that the increase of the temperature of step 4) compared to the temperature of step 3) improves cell viability and recovery. In case nucleation is induced by other methods as described above (e.g. by inducing a supercool spot), step 4) is preferably substituted by an incubation at the temperature that facilitates ice propagation throughout the medium (preferably the temperature of step 2)).

In step 5), the cooling rate of the ambient temperature to reach the at least −40° C. is slow, preferably up to 4.0° C. per minute, more preferably up to 2.0° C. per minute, most preferably up to 1.0° C. or even up to 0.5° C. per minute, e.g. about 0.3° C. per minute. Once about −40° C. is reached, or any temperature below, the cooling rate may be increased for convenience, e.g. to at least about 10° C. per minute, preferably at least about 15° C. per minute, or more preferably at least about 20° C., e.g. about 25° C. per minute. While the upper limit of the rate is not critical, it is envisaged that it should be about 40° C. per minute. While the temperature can be dropped suddenly, e.g. as described for step 3), or immediately (e.g. by placing the medium in a liquid nitrogen tank), it is preferred to do so only from temperatures below about −78° C., preferably below about −120° C., more preferably below about −150° C. In a preferred embodiment, the temperature cooled down to in step 5) is at least about −78° C., at least about −120° C., at least about −135° C., at least about −150° C., or at least about −160° C.

Definitions given and embodiments described with respect to the first, second, third and fourth aspect apply also to the fifth aspect, in as far as they are applicable. Also, definitions and embodiments described below, in particular under the header ‘Definitions and further embodiments’ apply to the fifth aspect.

In a sixth aspect, the invention relates to a cryoprotective medium comprising a pancreatic lineage cell cluster, wherein the cryoprotective medium comprises at least about 1% v/v EG and at least about 1% v/v DMSO. Preferred embodiments of cell cluster and of concentrations are described with regard to the method of the fifth aspect. The medium may also be described as a basal cryoprotective medium as defined above, supplemented with EG and DMSO.

The invention also relates to a kit comprising the cryoprotective medium of the sixth aspect. In a preferred embodiment, the kit comprises
(i) a first cryoprotective medium of the sixth aspect comprising a first concentration of EG and DMSO as described above, and
(ii) a second cell cryoprotective medium of the sixth aspect comprising a second concentration of EG and DMSO as described above, and/or a third cell cryoprotective medium of the sixth aspect comprising a third concentration of EG and DMSO as described above.
   This kit is particularly useful for the method of the fifth aspect.

The invention also relates to the use of the cryoprotective medium or of the kit for cryoprotecting a pancreatic lineage cell cluster.

Definitions given and embodiments described with respect to the first, second, third, fourth and fifth aspect apply also to the sixth aspect, in as far as they are applicable. In addition, definitions and embodiments described below, in particular under the header ‘Definitions and further embodiments’ apply to the sixth aspect.

In a seventh aspect, the invention relates to a frozen cell culture comprising a plurality of pancreatic lineage cell clusters, characterized in that at least 20% of the cells (in the culture) are viable. Viability is determined after thawing. In other words, at least 20% of the cells are recoverable by thawing. Preferably, at least 30%, 40%, 50%, 60%, 70%, 80% or 90% of the pancreatic lineage cells are viable (recoverably by thawing). These percentages relate to all cells that were frozen, i.e. all cells in the frozen culture (of which some may have been damaged by freezing), so not all cells in the frozen cell culture are necessarily viable. As such, the percentages represent the ratio of viable cells prior to freezing and viable cells after thawing. It is to be understood that clustering of the cells remains after thawing.

In a preferred embodiment, the medium of the frozen cell culture is the cryoprotective medium of the sixth aspect, preferably with the final concentrations of EG and DMSO as described above. In an even more preferred embodiment, the frozen cell culture is obtainable by the method of the fifth aspect.

It is further preferred that the pancreatic lineage cell clusters are endocrine progenitor cell clusters. Therein, it is preferred that the endocrine progenitor cell clusters are within about 1 to about 3 days, preferably within 1 to about 2 days more preferably within about 1 day after reaching the endocrine stage (i.e. were frozen at that time).

Definitions given and embodiments described with respect to the first, second, third, fourth, fifth and sixth aspect apply also to the seventh aspect, in as far as they are applicable. Also, definitions and embodiments described below, in particular under the header ‘Definitions and further embodiments’ apply to the seventh aspect.

In an eighth aspect, the invention relates to a method of producing a thawed pancreatic lineage cell cluster, comprising the steps of
(i) providing a frozen pancreatic lineage cell cluster, and
(ii) thawing the frozen pancreatic lineage cell cluster.

In a preferred embodiment, the frozen pancreatic lineage cell cluster provided in step (i) is obtainable by the method of the fifth aspect (preferably the step comprises the method of the fifth aspect) or is a frozen pancreatic lineage cell cluster as described to be comprised in a culture of the seventh aspect, and/or step (ii) comprises contacting the pancreatic lineage cell cluster with a ROCK inhibitor.

Step (ii) preferably comprises thawing the frozen pancreatic lineage cell cluster at an ambient temperature between about 10° C. and about 50° C., preferably between about 20° C. and about 45° C., more preferably between about 30° C. and about 40° C., most preferably at about 37° C. In a preferred embodiment, after the ice has started melting and before all ice has melted, preferably when most ice has melted (e.g. more than 60%, 70%, 80% or 90%), a thawing medium is added, preferably not at once, e.g. dropwise. The thawing medium may comprise as supplements a chemically defined serum-free formulation suitable for growing and maintaining cells, such as pluripotent cells, and preferably a ROCK inhibitor. Preferably, the formulation comprises amino acids, trace elements, vitamins/antioxidants, and proteins, as described and exemplified for the basal cryoprotective medium in the fifth aspect above.

These supplements aside, the thawing medium is a medium suitable for maintaining the pancreatic lineage cell cluster. In one embodiment, it may be a differentiation medium suitable for differentiating a precursor cell cluster of the pancreatic lineage cell cluster into the pancreatic lineage cell cluster. For instance, for an endocrine progenitor cell cluster, the thawing medium is a differentiation medium suitable for differentiating a pancreatic progenitor cell cluster into an endocrine progenitor cell cluster. For instance, it may be a basal medium as described above comprising an inhibitor of BMP signaling, a hedgehog signaling pathway inhibitor (preferably a Smoothened antagonist such as SANT1, SANT2, Cyclopamine or IHR1), an Alk5 inhibitor, a retinoic acid receptor agonist such as retinoic acid or EC23, thyroid hormone or a functional equivalent thereof as described above, supplements including antioxidant enzymes, proteins, vitamins, and fatty acids (e.g. B27 supplement), zinc, a polyanionic polymer, and/or supplements reducing the need for fetal bovine serum. Examples and preferred embodiments of these components are indicated above. Preferably before all ice has melted (e.g. when about 5% to about 25% of the ice remains), and more preferably when the medium temperature surrounding the remaining ice has reached about 3 to about 12° C., e.g. 5° C., thawing medium (preferably comprising the defined serum-free formulation and more preferably the ROCK inhibitor) may be added. Once the medium reaches room temperature (without or preferably with the addition of the thawing medium), the medium can be replaced with such thawing medium. Replacing medium is preferably without centrifugation and can be achieved by letting the cell cluster settle down by gravity at the container bottom and removing the supernatant. Preferably, a plurality of thawed cell clusters are obtained with the method and are seeded at a lower cell concentration. In a preferred embodiment, the cell cluster(s) is (are) maintained in the thawing medium for about 6 to about 48 hours, preferably about 12 to about 36 hours, more preferably about 18 to about 30 hours and most preferably about 24 hours. The thawed cell cluster(s) may be maintained under constant agitation for any amount of time, e.g. up to about 3 months, up to about 1 month or up to about two weeks. When the thawed cell cluster(s) are intended to be differentiated as soon as possible after thawing, it is preferred that they are maintained under constant agitation e.g. for about 3 to about 6 days or about 4 to about 5 days. During maintenance, it is preferred that the medium is changed for fresh medium at least once in about 2 days, preferably at least once in about 72 hours.

Optionally, the method further comprises a step (iii) of differentiating the thawed cell cluster, preferably using a cell medium of the fourth aspect, more preferably the first cell medium of the fourth aspect and the second cell medium of the fourth aspect, most preferably according to the method of the first aspect.

The method may further comprise a step at any stage after thawing, comprising determining viability and/or functionality of the cells in the cluster, preferably of a sample of a population of cell clusters, wherein the sample comprises at least one cell cluster. This allows confirming the suitability of the cell clusters in the population for the intended use (e.g. further differentiation, maturation, implantation etc). This can be accomplished using a variety of methods known in the art. For example, the cells can be stained using vital stains and counterstains, such as, e.g., fluorescein diacetate (FDA) and trypan blue, or propidium iodide (PI). It is envisaged that, for example, a population of islet-like clusters suitable for implantation comprises between about 20-100% viable cells, preferably at least about 50%, 55%, 60%, 65%, 70%, 75%, 80%, 85%, 90%, 95%, 96%, 97%, 98%, or most preferably at least about 99%, viable cells. In other aspects of the present invention, the morphometric characteristics of the cells can be determined as a measure of the suitability of cells for the intended use, e.g., in transplantation. Viability can, for instance, be determined by obtaining a sample of cell clusters prior and after cryopreservation, clusters from both samples can be dissociated into single cells, and single cells can be counted. By comparing the cell numbers obtained before and after freeze/thaw and recovery, a value for percent recovery can be determined.

Definitions given and embodiments described with respect to the first, second, third, fourth, fifth, sixth and seventh aspect apply also to the eighth aspect, in as far as they are applicable. Also, definitions and embodiments described below, in particular under the header ‘Definitions and further embodiments’ of the invention apply to the eighth aspect.

In a ninth aspect, the invention relates to a thawed cell culture comprising a plurality of pancreatic lineage cell clusters, wherein the thawed cell culture is obtained from a culture of frozen cells (meaning frozen cell culture) and the recovery of viable cells is at least 20% of the frozen cells. In other words, the invention relates to a thawed cell culture comprising a plurality of pancreatic lineage cell clusters, wherein at least 20% of the cells that were thawed (and previously frozen) are viable. Preferably, at least 30%, 40%, 50%, 60%, 70%, 80% or 90% of the cells that were thawed (and previously frozen) are viable. The frozen cells comprise all cells that were frozen; not necessarily all of the cells having undergone the freezing are viable, i.e. the percentages represent the ratio of viable cells prior to freezing and viable cells after thawing, as explained with regard to the seventh aspect.

It is further preferred that the pancreatic lineage cell clusters are endocrine progenitor cell clusters. Therein, it is preferred that the endocrine progenitor cell clusters are within about 1 to about 3 days, preferably within 1 to about 2 days more preferably within about 1 day after reaching the endocrine stage (i.e. were frozen at that time).

In a preferred embodiment, the thawed cell culture is obtainable by the method of the eight aspect.

Definitions given and embodiments described with respect to the first, second, third, fourth, fifth, sixth, seventh and eighth aspect apply also to the ninth aspect, in as far as they are applicable. Also, definitions and embodiments described below, in particular under the header ‘Definitions and further embodiments’ of the invention apply to the ninth aspect.

In a tenth aspect, the invention relates to the cells (in particular the islet-like clusters) of cell culture of the second aspect for use as a medicament.

The use is generally as a transplant. In a preferred embodiment, the use is as insulin-providing medicament. More preferably, the use is for treating diabetes, in particular type I or II diabetes, MODY (Maturity Onset Diabetes of the Young, also known as type 3a diabetes), pancreatitis- or surgery-induced diabetes, or other types of diabetes requiring partial or full dependence on exogenous insulin.

The subject to be treated preferably is of the same species as the cells of the cell culture. Most preferably, the species is human. The subject may be immunosuppressed and/or the use may further comprise immunosuppressive treatment. This can prevent allo- and-/or autoimmunity-related rejection.

The use as a medicament may comprise direct infusion into the liver, e.g. via the portal vein, or implantation into the body of the subject at other locations (e.g. at subcutaneous, intramuscular, or peri-hepatic sites, into adipose tissue, at or near the peritoneum, or adjacent to digestive organs). Delivery of the cells may involve prevascularization of the implantation site.

Alternatively or additionally, the cells may be delivered in a device, capsule or mesh. Such devices, capsules or meshes may be open (thereby allowing vascularization) or closed such that the cells are immune-isolated (thereby reducing or eliminating the need for immunosuppressive treatments). Devices, capsules or meshes may involve components mediating active release or supply of oxygen to improve survival and/or long-term function of the cells in the subject. Devices, capsules or meshes may be treated or coated, or release active factors to improve vascularization and/or reduce fibrosis.

The cells of the cell culture may also be coated using polymers such as alginate or alginate derivatives, or using materials capable of preventing immune-mediated cell loss.

The cells of the cell culture may also be made less vulnerable to allo- and autoimmune-rejection by pharmacological treatment (e.g. by pulsed treatment with interferon gamma), and/or by genetic modifications (“cloaking” or “hypoimmune” modifications), which suppress allograft-directed and autoimmunity-mediated cell killing. This is possible by using suitable genetic modifications such as gene knockouts and/or overexpression. For example, such genetic modifications may comprise a HLA-I A/B/C or B2M (β2m) knockout to suppress T-cell killing of the cells, and/or genetic modifications suppressing NK-cell-, macrophage- and/or complement-mediated killing of the cells.

The cells may also be genetically modified to enhance long-term function and survival of in response to non-immunity related stress, such as metabolic stress in patients with type 2 diabetes. For example, this may involve overexpression of function-enhancing genes, and/or genetic knockout of stress-induced and/or survival- or function-suppressing genes.

Definitions given and embodiments described with respect to the first, second, third, fourth, fifth, sixth, seventh, eighth and ninth aspect apply also to the tenth aspect, in as far as they are applicable. Also, definitions and embodiments described below, in particular under the header ‘Definitions and further embodiments’ apply to the tenth aspect.

### Definitions and Further Embodiments

It is to be understood that all methods described herein are in vitro methods, unless indicated otherwise.

The terms “^{pos}” and “^{neg}” with respect to cell markers mean the presence and absence of expression, respectively, of the markers. The terms “positive” and “negative”, “+” and “−” or “⁺” and “⁻” can be used instead.

A “spheroid” or “ellipsoid” is a sphere flattened at the poles. Herein, the term refers to a sphere-like but not perfectly spherical body. Preferably, a spheroid has a shape that is substantially spherical.

The term “cryoprotection” refers to the protection of cells from damage during freezing, or in other words the increase in viability of cells after thawing in comparison to cells which were not cryoprotected. It can be achieved by the addition of suitable substances (“cryoprotectants”), which are known in the art, to the cell medium. A “cryoprotective medium” is a medium comprising at least one cryoprotectant and is suitable for protection of cells from damage during freezing. In the context of the invention, this is achieved by the presence of EG and DMSO. However, further cryoprotectants may be present.

“Viability” is a measure of the number of living cells in a population. It is usually described as the ratio of living cells/total cells at one time. In the context of the invention, the term “recovery” is used, which refers to a ratio of living (viable) cells, wherein the ratio's denominator is the number of living cells prior to an event such as cryoprotection or freezing (and optionally thawing after freezing), and the ratio's numerator is the number of living cells after the event.

A “suitable basal medium”, where referred to herein, is a medium comprising amino acids, a sugar such as glucose, and ions (e.g. calcium, magnesium, potassium, sodium, and/or phosphate), which are essential for cell survival and growth. The basal medium may further comprise supplements reducing the need for fetal bovine serum, such as ITS (Insulin-Transferrin-Selenium), preferably ITS-X (Insulin-Transferrin-Selenium-Ethanolamine). In one embodiment, it does not comprise serum.

A “SMAD and MAPK signaling activator”, where referred to herein, is preferably a ligand of BMPR2 and BMPR1, more preferably a bone morphogenic protein, and most preferably BMP4.

An “inhibitor of BMP signaling”, where referred to herein, is preferably an Alk2/3 inhibitor such as LDN (herein referring in particular to LDN-193189), dorsomorphin, noggin, chordin, K02288 or LDN-212854.

Zinc, when used in a medium herein, can be used in any soluble form, e.g. in the form of ZnSO4.

The term “ambient temperature” refers to the temperature surrounding a container comprising cells. It can also be described as “chamber temperature”, wherein the chamber comprises the container. As such, it is not to be confused with the term “room temperature”. An ambient temperature is usually applied to bring the medium and cells in the container to the same temperature, e.g. when the container is maintained for an extended period of time (e.g. at least for one minute) at the ambient temperature, for example when it is referred to “incubation of cells or medium at that temperature”, but not always (e.g. not for inducing ice nucleation).

The term “room temperature” refers temperature a temperature of about 18° C. to about 26° C., preferably of about 20° C. to about 24° C.

The term “about” as used herein (unless defined otherwise) means±10%, preferably ±5%, more preferably ±2% and most preferably ±1%.

The polyanionic polymer may be a synthetic polymer, a naturally occurring polymer or a polymer derived from a naturally occurring polymer by modification and/or chemical or enzymatic fragmentation. The polyanionic polymer contains a plurality of anionic groups such as carboxylate and/or sulphate groups. More preferably, the polyanionic polymer is a sulphate group containing polymer. For example, the polyanionic polymer may be selected from sulphated saccharides, sulphated cyclodextrins, or sulphated synthetic polymers such as acrylic polymers, aromatic polymers, and/or polyalcohols. More particularly, the polyanionic polymer is selected from heparins or heparin derivatives, heparan sulphates, chondroitin sulfates, dextran sulphates, pentosan polysulphates or derivatives or combinations thereof. In the following, non-limiting examples of polyanionic polymers suitable for the invention are provided: Chemically modified heparin-derived oligosaccharides, heparin-like oligosaccharides, dextran sulphates, sulphated low molecular weight glycosaminoglycans, dextrin-2-sulphates, cellulose sulphates and naphthalene sulfonate polymer (e.g. PRO 2000), PAVAS (a co-polymer of acrylic acid with vinyl alcohol sulphate), the sulphonated polymer PAMPS [poly(2-acryl-amido-2-methyl-1-propanesulfonic acid](Mw e.g. approximately 7000-12000), Chondroitin sulphates, sulphated cyclodextrins, Laminarin sulphate, Polyglycerin sulphates, Pentosan polysulphates (PPS) and derivatives thereof such lactose-modified pentosan polysulphates, fractionated PPS/low molecular weight PPS, or Fucoidan. Low molecular weight heparin (LMWH) analogues such as Enoxaparin, Dalteparin, Fragmin, Nadroparin, Tinzaparin, Fondaparinux, Bemiparin, Reviparin, Ardeparin, Certoparin, and/or Parnaparin, e.g. Lovenox®, Fraxiparin®, Sandoparin® or Arixtra®, are additional examples of suitable polymers. They are obtained by fractionation and/or limited enzymatic or chemical digestion of heparin, and have an average molecular weight of preferably about 3000 to about 7000 Da. A preferred polyanionic polymer is heparin, a derivative or an analogue thereof, in particular heparin.

The term “applicable” herein means technically compatible.

The invention is described by way of the following examples which are to be construed as merely illustrative and not limitative of the scope of the invention.

### EXAMPLES

### Example 1: Additional Gamma Secretase Inhibitor Treatment in Absence of T3 During EN to ILC Transition Results in Increased Pancreatic Islet Endocrine Populations

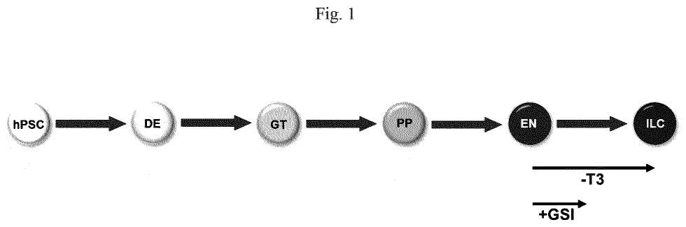

Beta cell differentiation from iPSC aggregates was carried out using several hiPSC lines with a new protocol derived by combining two published protocols (Nostro et al., Stem Cell Reports (2015), 4(4): 591-604; Rezania et al., Nat Biotechnol (2014), 32(11): 1121-1133), further referred to as “combination protocol”. iPSCs were seeded as single cells in suspension at 1×10⁶ cells/mL in mTeSR1 (StemCell Technologies Inc.; 05850) with 10 pM Y-27632 and kept in culture in 100 mL spinner flasks on a stirring platform to form aggregates under low shear conditions (see e.g. Fok and Zandstra, Stem Cells 2005; 23:1333-1342). At day 0 of differentiation, iPSC clusters were treated with Basal medium 1 (BM1) with the addition of 100 ng/mL Activin A, 2 μM CHIR 99021 and 10 μM Y-27632 to induce definitive endoderm. On subsequent days, media change was performed as described below, and occurred daily up to day 14, every other day after that. Definitive endoderm (DE) was induced from day 0 to day 3 and gut tube (GT) was patterned from day 3 to day 6. On day 6, pancreatic progenitors (PP) were induced until day 11. Commitment to the endocrine (EN) lineage was then performed between day 11 and day 14, and day 14 EN clusters were differentiated to islet-like clusters (ILCs) until day 24 (FIG. 1).

### Basal Media Used in Differentiation Experiments:

BM1: MCDB-131 (Life Technologies, 10372-019); 1× GlutaMAX (life Technologies, 35050-038); 1:5000 ITS-X (Life Technologies, 51500-056); 7.5 mM Glucose (Sigma, G7528); 0.1% rHSA (Biorbyt, orb419911); 2.5 g/L NaHCO₃(Roth, 6885.1); 1.5% Pen/Strep (Gibco, 15140-122).
BM2: MCDB-131 (Life Technologies, 10372-019); 1× GlutaMAX (life Technologies, 35050-038); 14.5 mM Glucose (Sigma, G7528); 2.5 g/L NaHCO₃(Roth, 6885.1); 1.5% Pen/Strep (Gibco, 15140-122).
BM3: MCDB-131 (Life Technologies, 10372-019); 1× GlutaMAX (life Technologies, 35050-038); 7.5 mM Glucose (Sigma, G7528); 0.1% rHSA (Biorbyt, orb419911); 1×NEAA (Life Technologies, 11140-35); 1.5% Pen/Strep (Gibco, 15140-122).
Day 0: BM1; 100 ng/mL Activin A (R&D Systems, 338-AC); 2 μM CHIR 99021 (Tocris, 12A/237643); 10 μM Y-27632 (Selleckchemicals, S1049)
Day 1-2: BM1; 100 ng/mL Activin A; 5 ng/mL bFGF (R&D systems, 233-FB/CF); 10 ng/mL Heparin (Sigma, H3149)
Day 3-5: BM1; 50 ng/mL FGF10 (R&D systems, 345-FG), 50 ng/mL Noggin (R&D Systems, 3344-NG); 10 μg/mL Heparin; 0.5 mM Vitamin C (Sigma, A4544)
Day 6: BM1; 2 μM RA (Sigma, R2625); 50 ng/mL FGF10, 50 ng/mL Noggin; 0.25 μM SANT1 (Sigma, S4572); 10 μg/mL Heparin; 0.25 mM Vitamin C; 1% B27-Vit A supplement (Gibco, 12587-010)
Day 7-10: BM1; 50 ng/mL Noggin; 50 ng/mL EGF (R&D Systems, 236-EG); 10 μM Nicotinamide (Sigma, N0636); 500 nM PDBu (Merck, 524390); 0.25 mM Vitamin C; 1% B27-Vit A supplement
Day 11-13: BM2; 0.1 μM LDN (Sigma, SML0559); 0.25 μM SANT1; 10 μM ALK5i II (Enzo, ALX-270-445); 50 nM RA; 1 μM T3; 0.25 mM Vitamin C; 0.5×B27 supplement (Gibco, 17504-044); 10 μM ZnSO4 (Sigma, 32047); 10 μg/mL Heparin, 1:200 ITS-X (Life Technologies, 51500-056)
Day 14-19: BM3; 0.1 μM LDN; 10 μM ALK5i II; 1 μM T3 (Millipore, 64245); 10 μM ZnSO4; 1:200 ITS-X
Day 20-24: BM3; 0.1 μM LDN; 10 μM ALK5i II; 1 μM T3; 10 μg/mL Heparin; 10 μM ZnSO4; 1:200 ITS-X
   The combination protocol was modified, from day 14 onwards, as follows:
Day 14-17: BM3; 0.1 μM LDN; 10 μM ALK5i II; 1 μM Gamma secretase inhibitor XXi (Millipore, 565790); 10 μM ZnSO4; 1:200 ITS-X
Day 18-24: BM3; 0.1 μM LDN; 10 μM ALK5i II; 10 μg/mL Heparin; 10 μM ZnSO4; 1:200 ITS-X
   This alternative to the combination protocol is referred to as “combination protocol +GSI/−T3”.

In addition, a further differentiation protocol (referred to as “second protocol”) was used to show that improvements by +GSI/−T3 are not limited to the combination protocol. As the combination protocol, the second protocol starts with seeding and aggregation of single pluripotent cells in suspension, and proceeds through the same intermediate stages of pancreatic embryonic development (definitive endoderm/DE, primitive gut tube, pancreatic progenitors, endocrine differentiation) to beta cell-containing islet-like clusters (ILCs) containing C-peptide^{pos}/insulin^{pos}/NKX6.1^{pos} beta cells. Differences to the combined protocol include the addition of a combination of BMP4 and bFGF during the first day of DE induction (see also Example 4), and longer durations of media incubations during pancreatic progenitor and endocrine induction, resulting in an overall longer time from start of differentiation to EN stage (EN stage on day 16 instead of day 14). Similar to the combined protocol, the second protocol was modified by additional gamma secretase inhibitor treatment in absence of T3 during the EN to ILC transition. The latter version of the adapted protocol is referred to “second protocol +GSI/−T3”.

The inventors confirmed that gamma secretase inhibitor XXI can be replaced by gamma secretase inhibitor LY411575.

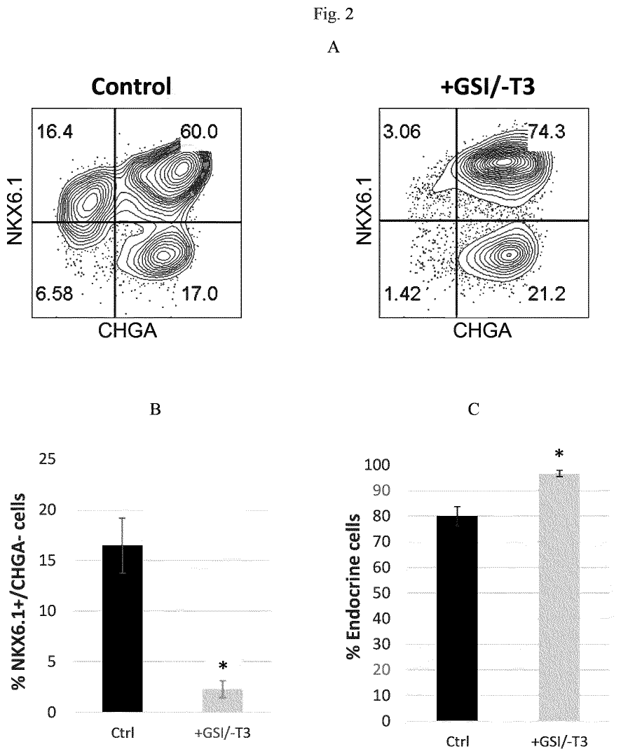

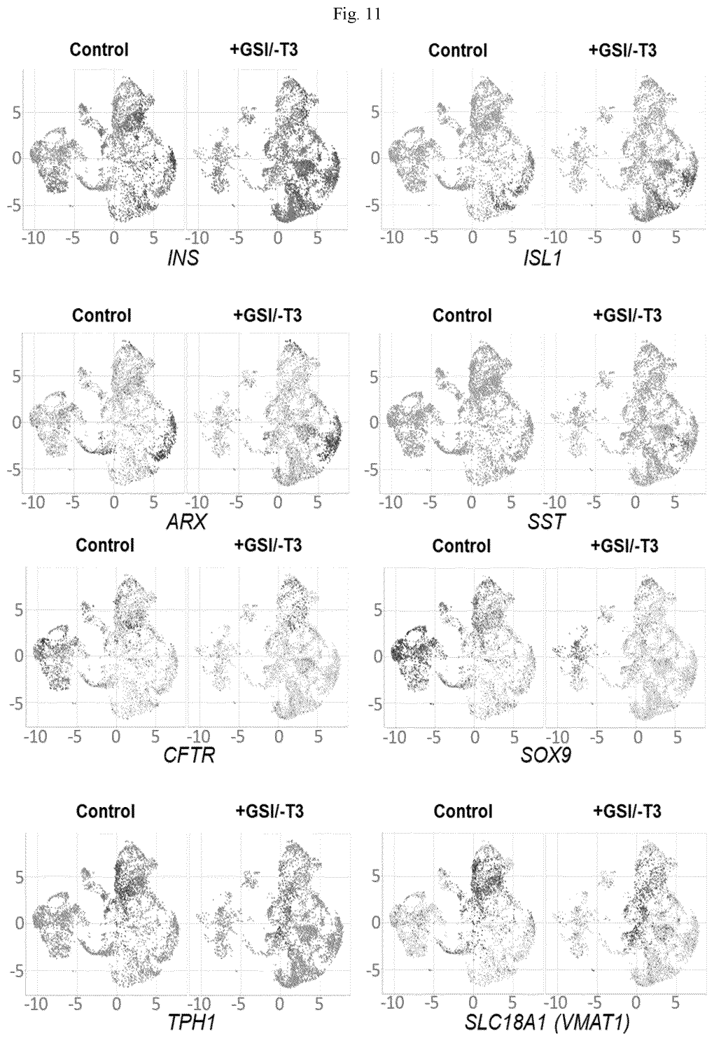

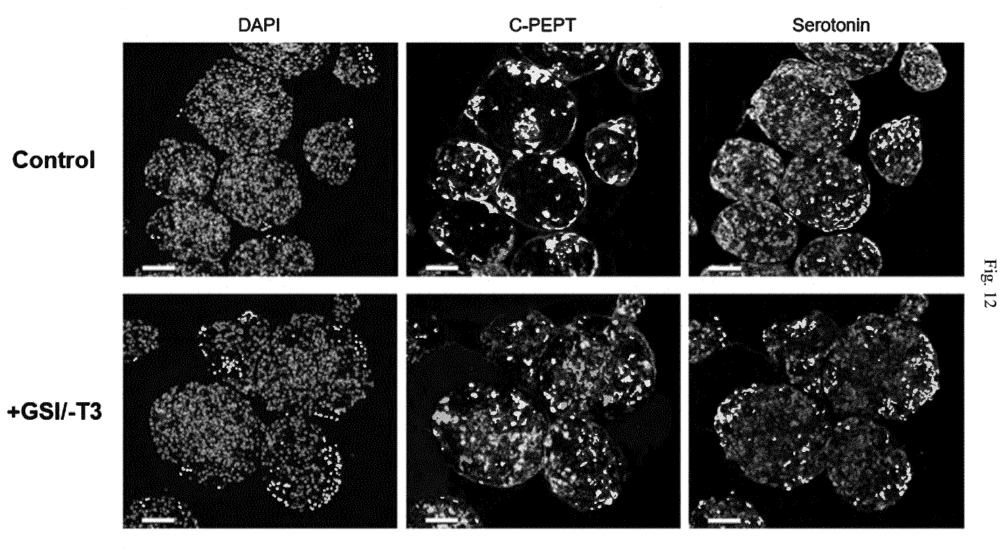

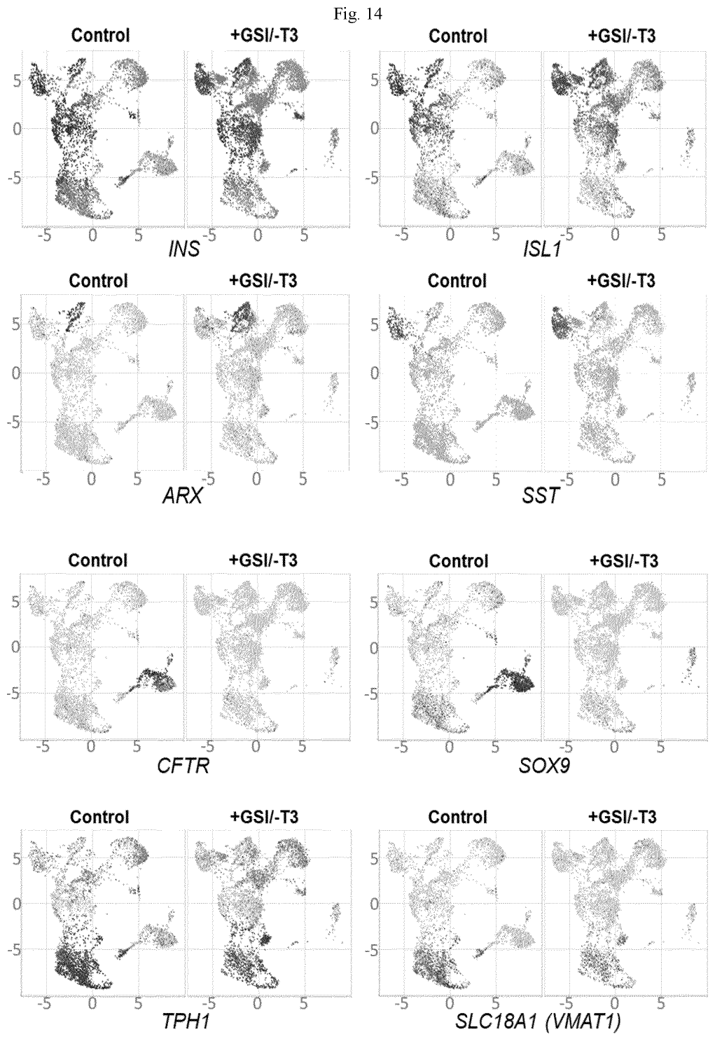

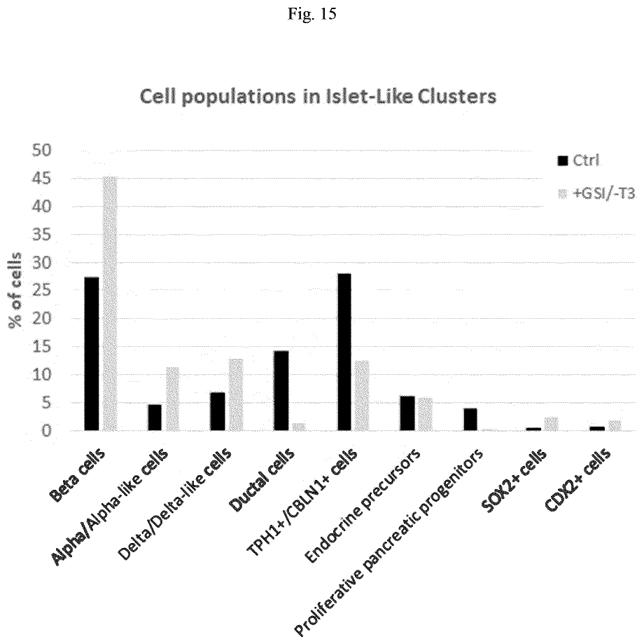

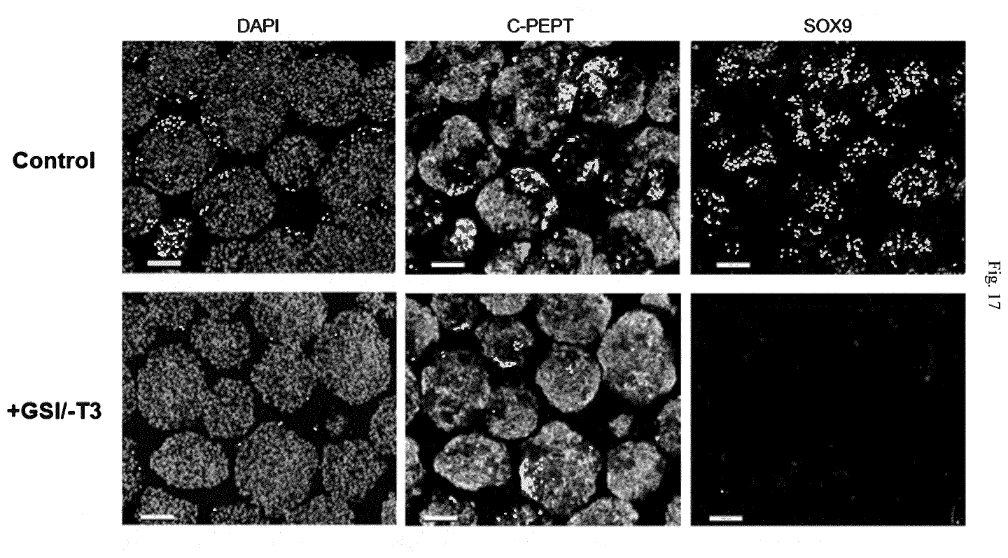

The addition of gamma secretase inhibitor and removal of T3 after EN stage efficiently blocked the ductal fate and significantly increased the endocrine population. As a result, the content of chromogranin A^{pos} cells rose to levels above 95% (FIGS. 2A, 2C). Only residual ductal cells were observed in islet-like clusters generated with the addition of gamma secretase inhibitor and removal of T3, as measured by single cell RNA sequencing (FIG. 7; FIG. 11; FIG. 14, ductal SOX9^{pos}/CFTR^{pos} populations; FIG. 15A) and histology (FIG. 13; FIG. 17).

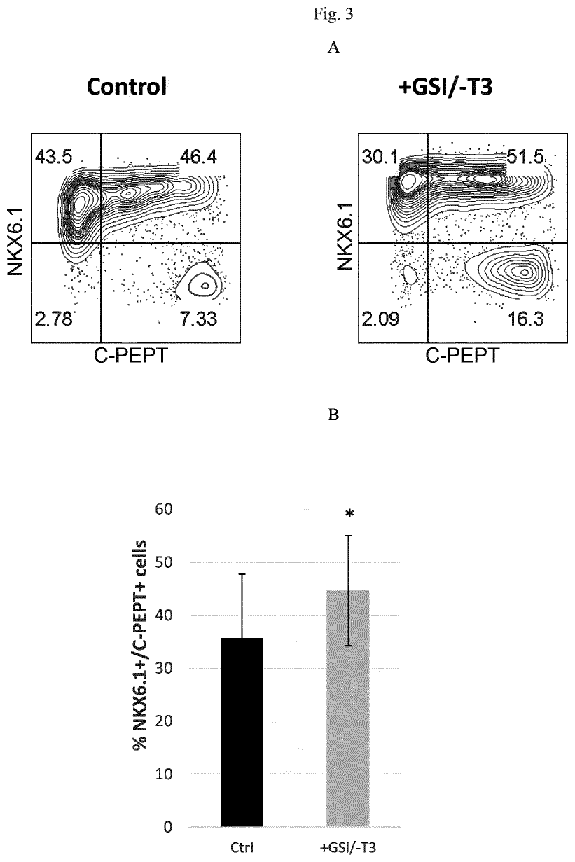

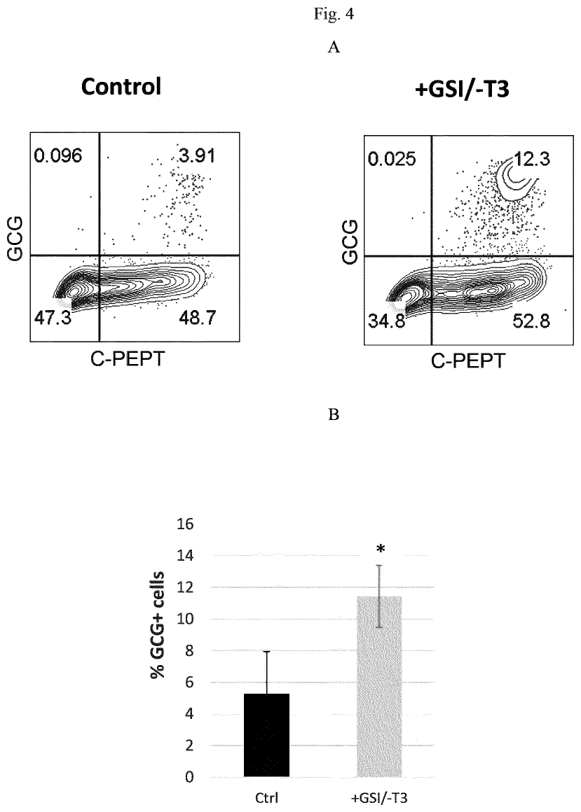

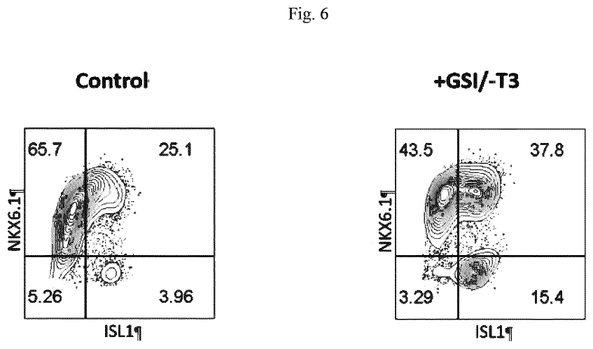

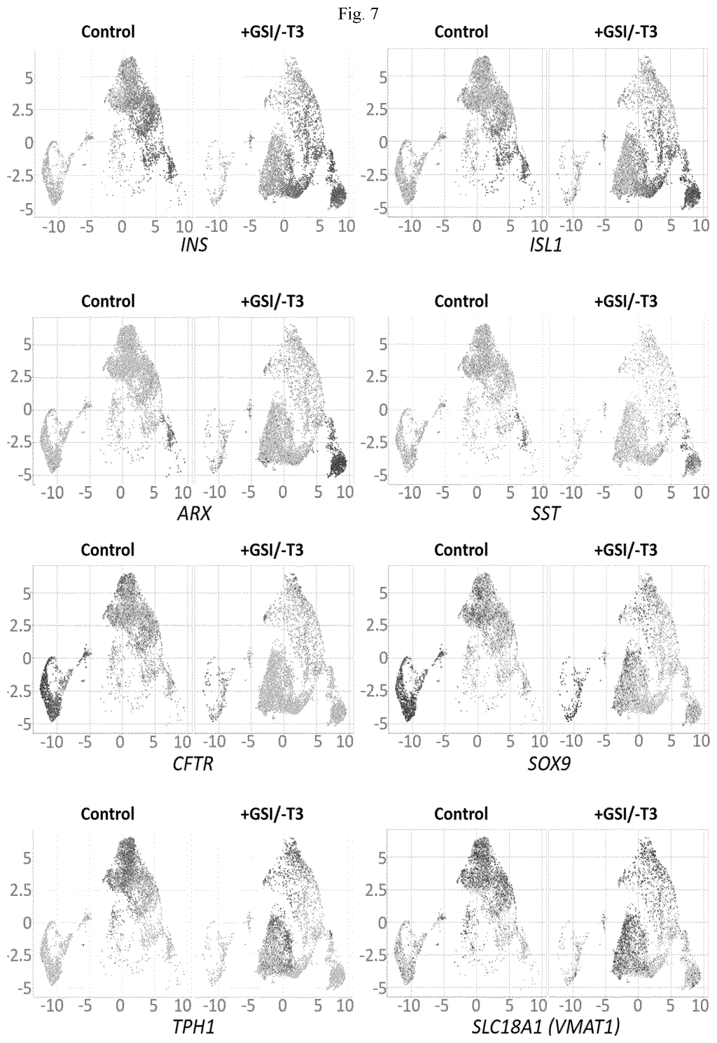

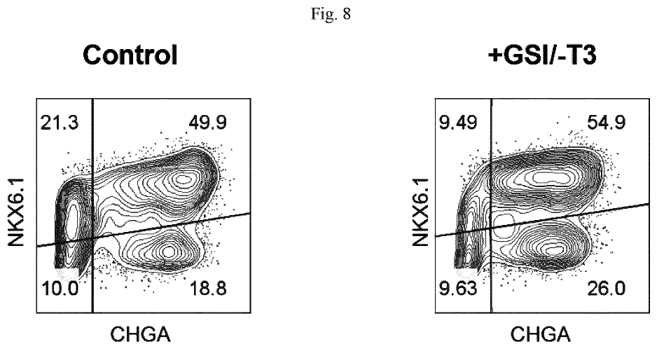

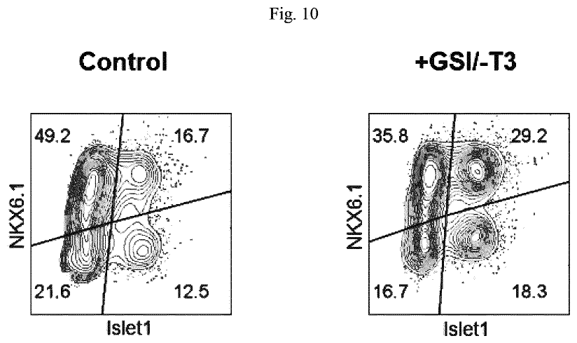

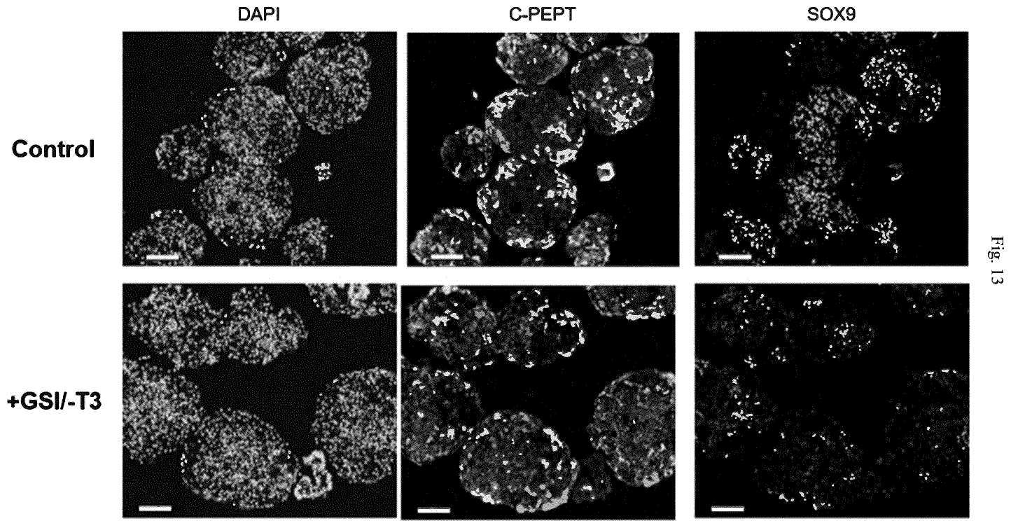

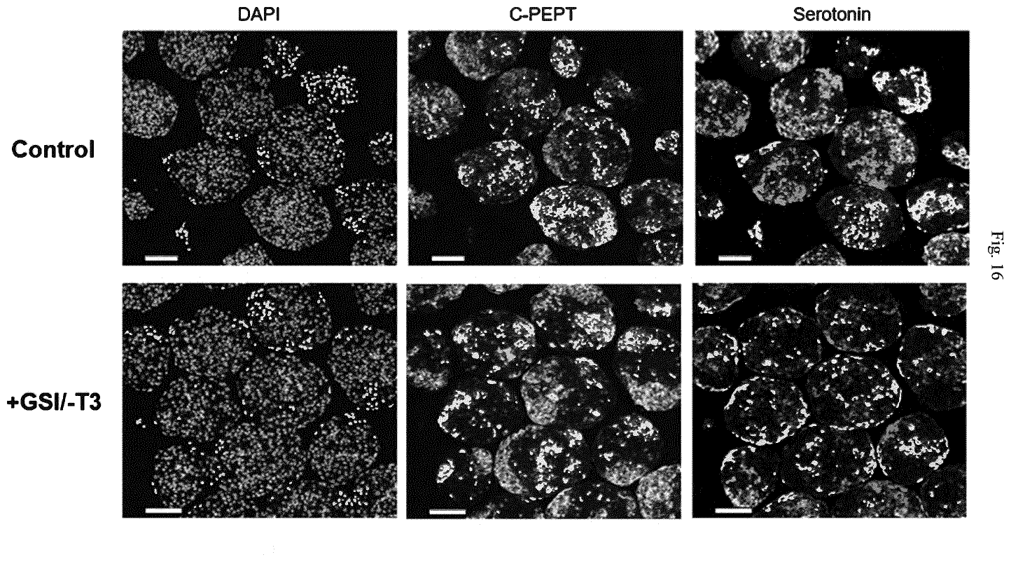

The addition of gamma secretase inhibitor and removal of T3 led to increased populations of pancreatic islet endocrine cells in islet-like clusters. The beta cell population was improved, as seen by FACS for NKX6.1^{pos}/C-peptide^{pos} (FIGS. 3A, B), that could reach levels >65%, FACS for NKX6.1^{pos}/ISL-1^{pos} (FIG. 6; FIG. 10), single cell RNA sequencing (FIG. 7; FIG. 11; FIG. 14; FIG. 15), and histology for C-peptide (FIG. 12; FIG. 13; FIG. 16; FIG. 17). Significantly higher numbers of glucagon^{pos} cells were also observed (FIGS. 4A, 4B), and the overall ARX-expressing pre-alpha cell population was larger compared to control conditions (FIG. 7; FIG. 11; FIG. 14). In addition, the somatostatin^{pos} population was increased in islet-like clusters generated with the addition of gamma secretase inhibitor and removal of T3 (FIG. 7; FIG. 11; FIG. 14; FIG. 15A).

### Example 2: Additional Gamma Secretase Inhibitor Treatment in Absence of T3 During EN to Islet-Like Cluster Transition Results in a Reduction of Pancreatic EC-Like Cell Content

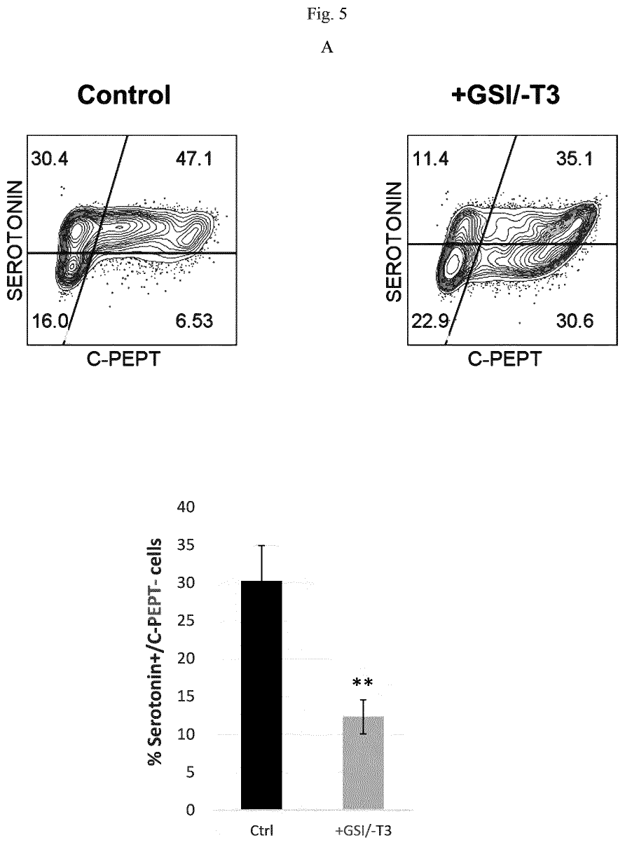

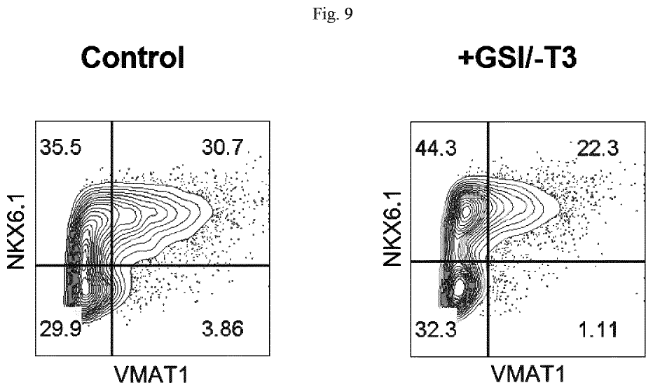

Pancreatic EC cells (also referred to simply as EC cells herein) produce serotonin but do not express insulin. Quantification of the EC cell population in islet-like clusters was performed by FACS for serotonin^{pos} and C-peptide^{neg} cells. While islet-like clusters generated using the combination protocol (Control) contain 30% of pancreatic EC cells, the addition of gamma secretase inhibitor in absence of T3 (+GSI/−T3) led to a drastic reduction of this population, down to levels <15% (FIGS. 5A, 5B). Histological analysis of islet-like clusters showed that while clusters generated with the unmodified versions of either the combination or second protocol (Control) displayed large patches of bright serotonin-expressing cells, these patches were much smaller and with faint serotonin-expression, and with only scarce bright serotonin-expressing cells in clusters generated with additional gamma secretase inhibitor in absence of T3 (+GSI/−T3) (FIG. 12; FIG. 16). VMAT1/SLC18A1 expression was found similarly reduced in islet-like clusters generated with the addition of gamma secretase inhibitor in absence of T3 (FIG. 9; FIG. 7; FIG. 11; FIG. 14).

### Example 3: Better In Vivo Performance from Islet-Like Clusters Generated with the Additional Gamma Secretase Inhibitor Treatment in Absence of T3 During EN to Islet-Like Cluster (ILC) Phase

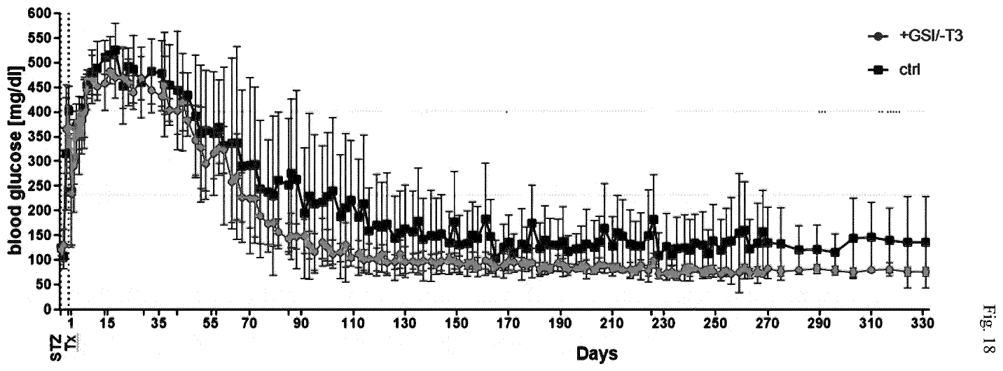

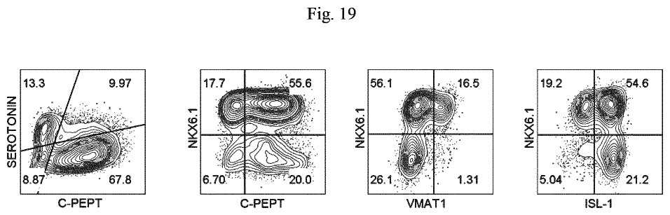

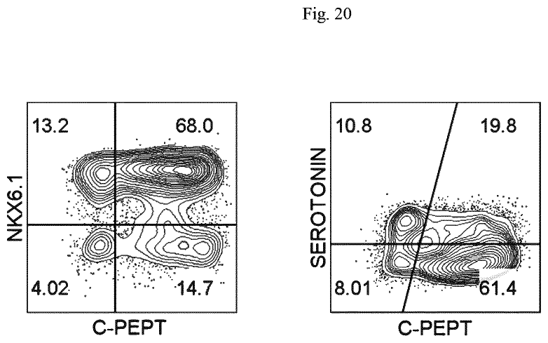

Islet-like clusters were generated with either the second protocol or the second protocol including additional gamma secretase inhibitor treatment in absence of T3 during EN to ILC transition phase, and functionally assessed in vivo for their ability to reverse diabetes in comparison to clusters generated under control conditions. FIG. 18 shows the blood glucose monitoring of Streptozotocin-treated NOD Scid Gamma mice transplanted with islet-like clusters under the kidney capsule. Islet-like clusters differentiated with additional gamma secretase inhibitor in absence of T3 (+GSI/−T3) were able to normalize blood glucose faster and maintained lower blood glucose levels than clusters generated with the second protocol (Control), reaching and maintaining the human blood glucose setpoint below 100 mg/dl.

### Example 4: BMP4 and bFGF Improve Definitive Endoderm (DE) Induction in Cell Lines with Suboptimal DE Induction

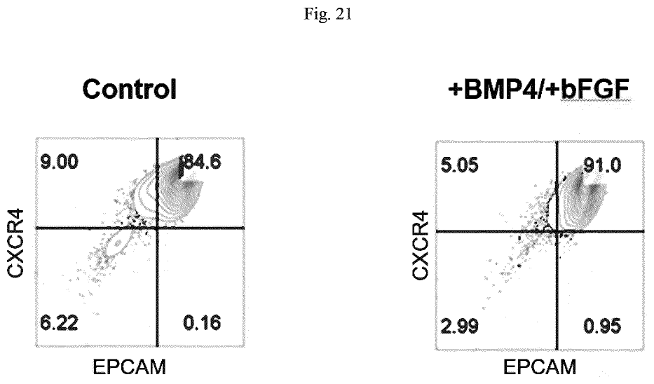

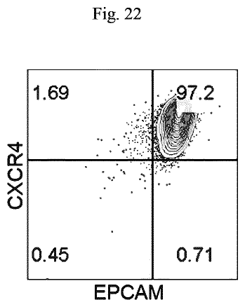

The efficiency of definitive endoderm induction can be assessed by the co-expression of CXCR4 and EPCAM. Definitive endoderm is mainly induced by high amounts of Activin A and enhanced with the activation of WNT signaling. An example is shown in FIG. 21. Control clusters generated according to the combination protocol (Control), which includes treatment with a combination of Activin A and CHIR99021 during DE induction, differentiated successfully and did not have residual pluripotent cells (CXCR4^{neg}/EPCAM^{pos}). However, a significant proportion of the cells developed into non-endodermal CXCR4^{pos}/EPCAM^{neg} and CXCR4^{neg}/EPCAM^{neg} populations. Addition of BMP4 and bFGF was able to prevent their development and improve DE induction to optimal levels (FIG. 21, +BMP4/+bFGF; FIG. 22, >97%).

### Example 6: Addition of GSI in the Absence of T3 Improves Cluster Composition of Post-Thaw Islet-Like Clusters and Non-Cryopreserved Islet-Like Clusters

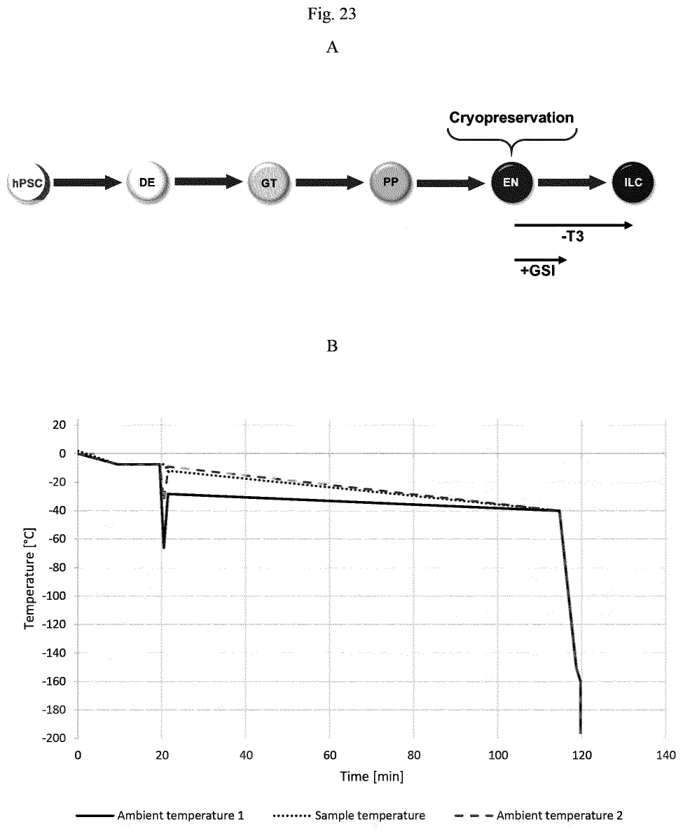

Using the combination protocol, induced pluripotent stem cells were differentiated into EN clusters. FIG. 23 shows a schematic overview of the differentiation process, cryopreservation and subsequent further differentiation. Clusters were cryoprotected before freezing by addition of the basal cryoprotective medium, consisting of KnockOut™ Serum Replacement, 10 μM ROCKi and 2 μM fatty acids, with increasing concentrations of DMSO and EG, in combination with incubation steps at decreasing temperatures. The DMSO and EG concentrations during the cryoprotection protocol and the corresponding stock concentrations, along with incubation times and temperatures, are given in table 1.

TABLE 1
Cryoprotectant concentrations during stepwise addition of cryoprotectant before freezing.

| | | Cryoprotectant concentrations [%] during | | |
|---|---|---|---|---|
| Final cryoprotectant | Cryoprotectant | stepwise protocol | | |
| concentration of used | concentration [%] of | Step 1: 5 | Step 2: 25 | Step 3: 15 |
| freezing media | used stock solution | min at 22° C. | min at 0° C. | min at 0° C. |
| 7.2% DMSO + 2.8% | 21.3% DMSO, | 2.3% DMSO, | 3.8% DMSO, | 7.2% DMSO, |
| EG (Medium A) | 11.2% EG | 0.9% EG | 1.5% EG | 2.8% EG |

EN clusters, transferred to cryogenic vials, were frozen directly after the stepwise cryoprotection treatment in a controlled rate freezer using the following protocol (freezing procedure A):
1. The sample is cooled-down to −7.5° C. (+/−0.7° C.);
2. Induction of nucleation by mechanical agitation and inducing a supercool spot at the outer vial wall;
3. Incubation for 15-minutes at −7.5° C.;
4. Cool-down of sample to −40° C. at a rate of 0.3° C. per minute;
5. Immersion of sample in liquid nitrogen;
6. Transfer to liquid N₂ tank for long-term storage.

After low-temperature storage of at least seven days, the EN clusters were thawed rapidly in a 37° C. waterbath and the cryoprotective medium is diluted with thawing medium, consisting of differentiation medium for the EN-stage as described previously, according to the combination protocol, supplemented with 20% KnockOut™ Serum Replacement and 10 μM ROCKi. After resuspension in fresh thawing medium, the EN clusters are cultured under standard culture conditions. The supplements in the thawing medium are removed by medium change after 24 hours, using differentiation medium for the EN-stage, according to the combination protocol, for further differentiation. Afterwards, the culture medium was refreshed regularly.

According to the combination protocol, EN clusters were treated without GSI and with T3 (Control) after thawing and compared to clusters incubated with GSI and without T3 after thawing, i.e. during the corresponding days of post-thaw differentiation.

Recovery rates were determined after post-thaw culture of 4-6 days, i.e. differentiation into islet-like clusters, by determination of viable cell count. Clusters, dissociated into single cells, were stained and analysed for different markers with flow cytometry.

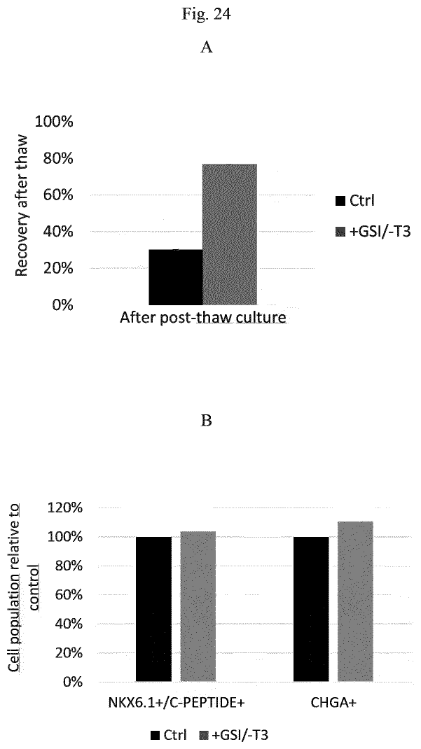

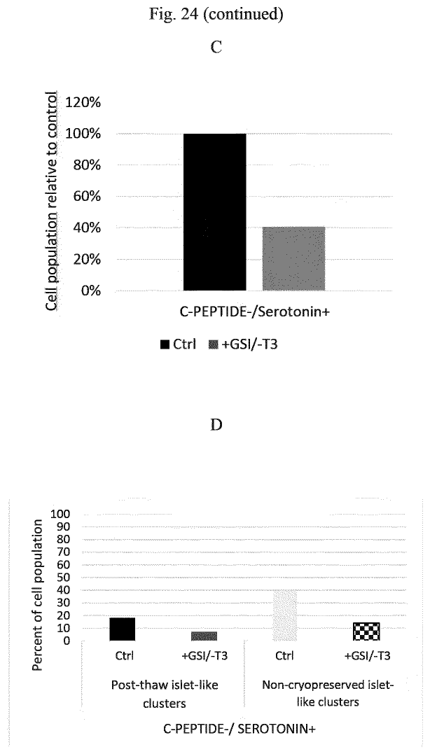

As shown in FIG. 24a, the addition of GSI, without T3 addition, increases the post-thaw recovery of the said cryopreserved islet-like clusters in comparison to the control.

As shown in FIG. 24b, addition of GSI, in the absence of T3, increases the number of beta-cells and Chromogranin^{pos} cells in the post-thaw islet-like clusters in reference to the control. As shown in FIG. 24c, the addition of GSI while omitting T3 decreases C-peptide^{neg}/serotonin^{pos} cell levels in post-thaw islet-like clusters relative to the control. As shown in FIG. 24d, the addition of GSI, in the absence of T3 treatment, decreases the population of C-peptide^{neg}/serotonin^{pos} cells in post-thaw islet like clusters and non-cryopreserved islet-like clusters in comparison to the respective controls.

### Example 6: Optimal Freezing During Differentiation into Islet-Like Clusters at Endocrine (EN) Stage

As described in the respective embodiments above, induced pluripotent stem cells were differentiated to EN clusters according to the second protocol, with extended GSI treatment in the absence of T3, and frozen at EN stage. The clusters were cryoprotected in a stepwise manner and frozen using 7.2% DMSO+2.8% EG and freezing procedure A, as described.

After storage, the EN clusters were thawed and cultured as described. Differentiation medium suitable for EN stage according to the second protocol with extended GSI in the absence of T3 addition was used for thawing and for further differentiation. Recovery rates were determined after post-thaw culture of 4-6 days, i.e. differentiation into islet-like clusters, by determination of viable cell count as described.

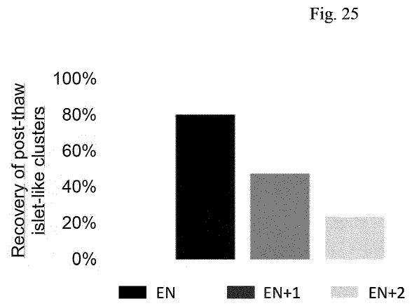

As shown in FIG. 25, the recovery rate decreases when EN clusters are frozen at later time points during the differentiation. Islet-like clusters have a higher recovery rate after thawing and post-thaw differentiation when frozen at the endocrine (EN) stage in comparison to subsequent days. Freezing clusters at EN stage and EN+1 (EN stage+1 day) yield the best recovery rates.

### Example 7: Generation of Beta- and Endocrine Cells is Comparable Between Post-Thaw Islet-Like Clusters and Non-Cryopreserved Islet-Like Clusters, Treated with Extended GSI without T3, with No Adverse Effects on Cell Composition

While applying the second differentiation protocol, clusters were frozen at EN+1 using the described cryoprotective medium of 7.2% DMSO+2.8% EG added within the stepwise cryoprotection treatment and freezing procedure A. After thawing the EN clusters as described, the clusters were differentiated into islet-like clusters according to the second protocol (Control) in comparison to the second protocol with extension of GSI and without T3 addition. Single-cell RNA sequencing was carried out after thawing and differentiation into islet-like clusters, after dissociation of clusters into single cells.

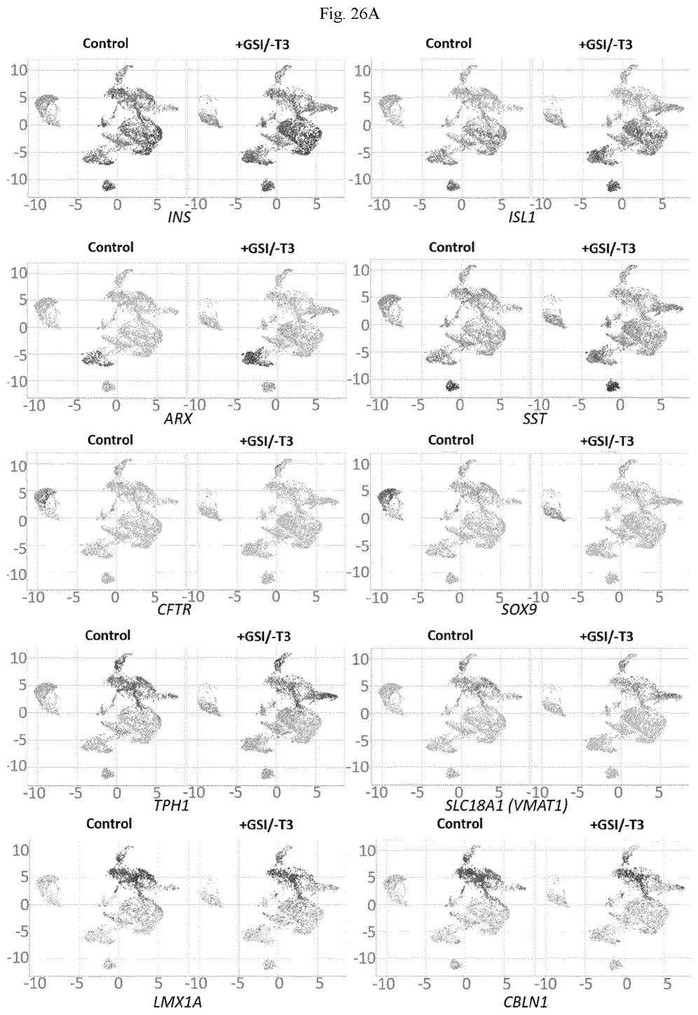

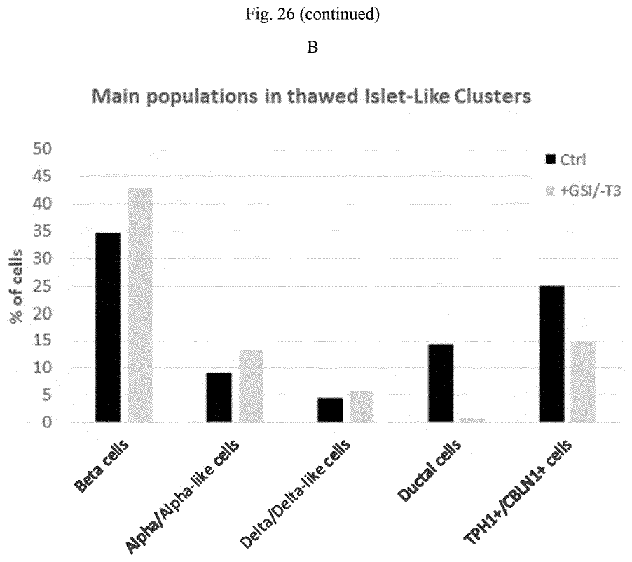

As shown in FIG. 26a, the extended addition of GSI, in the absence of T3, increases cell populations expressing INS, ISL-1, ARX and SST in comparison to cells without the extension of GSI with T3 addition. SOX9 and CFTR-expressing (ductal) cells are reduced by extending GSI addition in the absence of T3. TPH1, VMAT1, LMX1A and CBLN1 expressing cells, representing serotonin-expressing cells, are also reduced by GSI extension, without T3 addition. As shown in FIG. 26b, populations of beta-cells, populations of ARX^{pos} cells and somatostatin (SST)^{pos} cells are increased by extended addition of GSI, in the absence of T3 in comparison to the Ctrl. The population of ductal cells is decreased to low levels by addition of GSI without T3.

While applying the second differentiation protocol, with extended GSI in the absence of T3, clusters were frozen at EN+1 using the described cryoprotective medium of 7.2% DMSO+2.8% EG, added within the stepwise cryoprotection treatment and freezing protocol A. After thawing, the clusters were further differentiated into islet-like clusters, according to the described procedures and the second protocol, with extended GSI the absence of T3. Islet-like clusters, dissociated into single cells, were stained and analysed for different markers with flow cytometry.

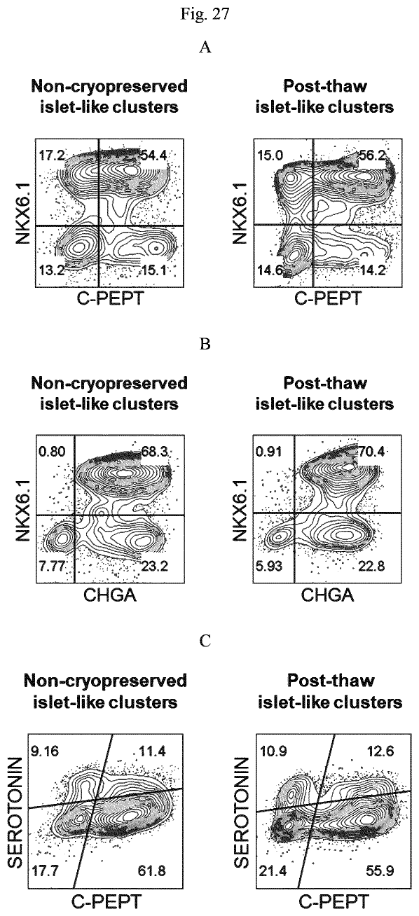

As shown in FIG. 27a, a NKX6.1^{pos}/C-peptide^{pos} cell population (beta-cells) is present in post-thaw islet-like clusters, which is comparable in size to the beta-cell population in the non-cryopreserved islet-like clusters.

As shown in FIG. 27b, the post-thaw islet-like clusters contain a NKX6.1^{pos}/Chromogranin^{pos} cell (endocrine cell) population, which is comparable in size to the endocrine cell population in the non-cryopreserved islet-like clusters at EN+7.

As shown in FIG. 27c, the amount of serotonin^{pos} cells in the post-thaw islet-like clusters, is comparable to the serotonin^{pos} cells in the non-cryopreserved islet-like clusters at EN+7. Levels of C-peptide^{neg}/serotonin^{pos} cells are similar in post-thaw islet-like clusters and non-cryopreserved islet-like clusters, demonstrating that cryopreservation has no adverse effects on cell composition.

### Example 8: Optimized Freezing with Cryoprotective Medium Containing 4% DMSO+6% EG

As described in the respective embodiments above, induced pluripotent stem cells were differentiated to EN clusters according to the second protocol, with extended GSI treatment in the absence of T3, and frozen at EN+1 stage.

The clusters were cryoprotected before freezing by addition of the basal cryoprotective medium, with increasing concentrations of DMSO and EG, in combination with incubation steps at decreasing temperatures. The DMSO and EG concentrations during the cryoprotection protocol and the corresponding stock concentrations, along with incubation times and temperatures, are given in table 2 for the respective final concentrations of DMSO and EG.

TABLE 2
Cryoprotectant concentrations during stepwise addition of cryoprotectant before freezing.

| | | Cryoprotectant concentrations [%] during | | |
|---|---|---|---|---|
| Final cryoprotectant | Cryoprotectant | stepwise protocol | | |
| concentration of used | concentration [%] of | Step 1: 5 | Step 2: 25 | Step 3: 15 |
| freezing media | used stock solution | min at 22° C. | min at 0° C. | min at 0° C. |
| 7.2% DMSO + 2.8% | 21.3% DMSO, | 2.3% DMSO, | 3.8% DMSO, | 7.2% DMSO, |
| EG (Medium A) | 11.2% EG | 0.9% EG | 1.5% EG | 2.8% EG |
| 4% DMSO + 6% EG | 12% DMSO, | 1.3% DMSO, | 2.2% DMSO | 4% DMSO, |
| | 24% EG | 2.0% EG | 3.3% EG | 6% EG |
| 4% DMSO + 4% EG | 12% DMSO, | 1.3% DMSO, | 2.2% DMSO | 4% DMSO, |
| | 16% EG | 1.3% EG | 2.2% EG | 4% EG |
| 4% DMSO + 7.5% EG | 12% DMSO, | 1.3% DMSO, | 2.2% DMSO, | 4% DMSO, |
| | 30% EG | 2.4% EG | 4.1% EG | 7.5% EG |

Subsequent to the cryoprotection, the clusters were frozen using freezing procedure A, as described. After low-temperature storage, the EN clusters were thawed and differentiated further as described, according to the second protocol. Recovery rates of post-thaw islet-like clusters were determined by determination of viable cell count. Viability was determined with a viability staining with PI and FDA, imaged with a fluorescence microscope.

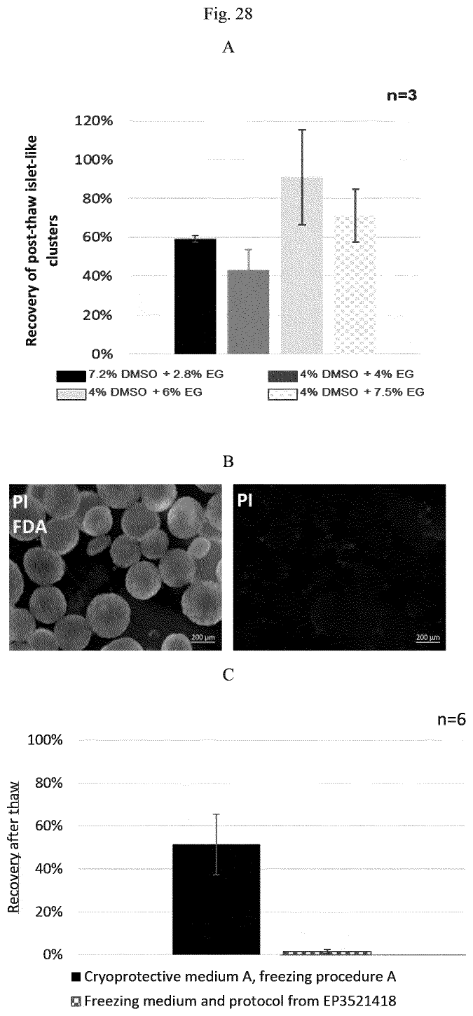

As shown in FIG. 28a, recovery rates of islet-like clusters are increased further by lower DMSO in combination with EG concentrations of 6% or above when using the described freezing procedure for freezing of clusters at EN+1. The usage of 4% DMSO with an EG concentration of 6% for cryoprotective media led to higher recovery rates of islet-like clusters.

As shown in in FIG. 28b, EN clusters frozen with 4% DMSO+6% EG for cryoprotective media have high viability on the day of thawing.

In comparison to the cryoprotective medium A with 7.2% DMSO+2.8% EG added within the stepwise cryoprotection protocol and the freezing procedure A, the EN clusters were frozen based on the following procedure taken from EP3521418:
Freezing medium: Dulbecco's Modified Eagle Medium (DMEM)+25 mM HEPES Buffer Solution+30% KnockOut™ Serum Replacement+10% DMSO,
Pre-freeze treatment: Pre-incubation for 20 min on ice,
Freezer protocol:
   1. The sample is cooled down from 0° C. to −9° C. at a rate of 2° C. per minute;
   2. Incubation at −9° C. for 10 min;
   3. Induction of nucleation by mechanical agitation and inducing a supercool spot at the outer vial wall;
   4. Incubation at −9° C. for 10 min;
   5. Cool-down from −9° C. to −40° C. at a rate of 0.2° C. per minute;
   6. Fast cool-down from −40° C. to −150° C. at a rate of 25° C. per minute;
   7. Storage in N₂ tank.

EN clusters frozen with the cryoprotective medium A, cryoprotection treatment and subsequent freezing with freezing procedure A were thawed and differentiated post-thaw into islet-like clusters according to the described methods. EN clusters frozen with methods described in EP3521418 were treated in the exact same manner during thaw and post-thaw differentiation. As seen in FIG. 28c, islet-like clusters generated from EN clusters frozen with the new procedure had higher post-thaw recovery than islet-like clusters generated from EN clusters frozen with methods described in EP3521418.

### Example 9: Highly Viable Islet-Like Clusters after Freezing Procedure Including Ambient Temperature Dip to Induce Nucleation and Extended GSI Treatment Post-Thaw

While using the second differentiation protocol, EN clusters were cryoprotected with the cryoprotective medium of 7.2% DMSO+2.8% EG in a stepwise manner as described, before freezing. The EN clusters were frozen with freezing procedure A in comparison to the following revised freezing procedure (temperature dip −33° C.). An example temperature curve for the ambient temperature and sample temperature is shown in FIG. 23.
1. The sample is cooled-down to −7.5° C. at a rate of 1.5° C. per minute;
2. Incubation at −7.5° C. for 10 minutes;
3. Drop in ambient temperature to −33° C. at a rate of 99° C. per minute;
4. Return to −9° C. ambient temperature with 72° C. per minute;
5. Cool-down to −40° C. at a rate of 0.3° C. per minute;
6. Immersion of sample in liquid nitrogen;
7. Transfer to liquid N₂ tank for long-term storage.

After low-temperature storage, the EN clusters were thawed as described and differentiated further into islet-like clusters. Recovery rates of post-thaw islet-like clusters were determined by determination of viable cell count. Viability was determined with a viability staining with PI and FDA, imaged with a fluorescence microscope. Islet-like clusters, dissociated into single cells, were stained and analysed for different markers with flow cytometry.

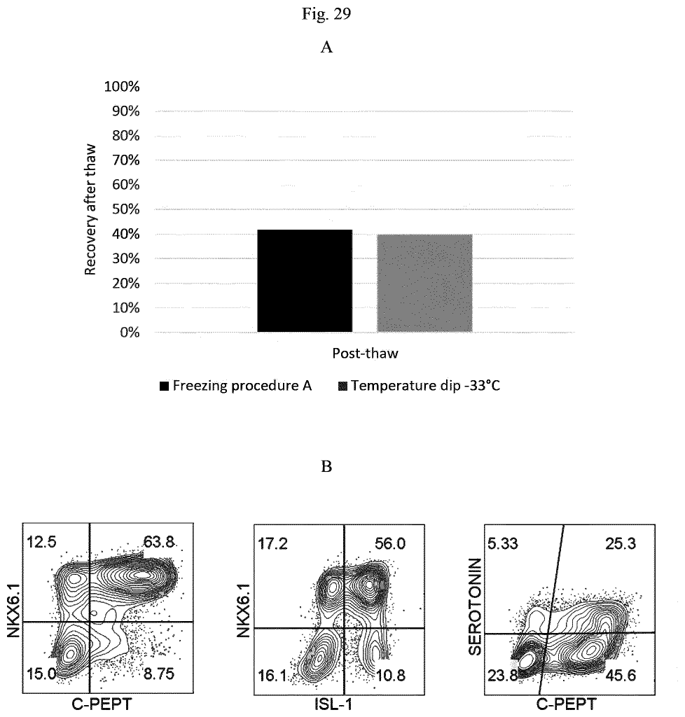

As shown in FIG. 29a, post-thaw recovery with the temperature dip to −33° C. to induce nucleation resulted in post-thaw recovery comparable to the post-thaw recovery with freezing procedure A. As shown in FIG. 29b, application of the said freezing procedure and post-thaw GSI treatment combined with omission of T3 treatment resulted in islet-like clusters with a high beta-cell population and low serotonin levels.

Using the combination differentiation protocol, induced pluripotent stem cells were differentiated into EN clusters. At the EN stage, the clusters were cryopreserved with 4% DMSO+6% EG and the described stepwise cryoprotection treatment. The following revised freezing procedure (temperature dip −33° C., then −28° C.) was used:
1. The sample is cooled-down from 0° C. to −7.5° C. at a rate of 1.5° C. per minute;
2. Incubation at −7.5° C. for 10 minutes;
3. Ambient temperature drop to −28° C. at a rate of 35° C. per minute;
4. Ambient temperature drop to −33° C. at a rate of 2.5° C. per minute;
5. Return to −28° C. ambient temperature with 2.5° C. per minute;
6. Cool-down of the sample to −40° C. at 0.3° C. per minute;
7. Decrease of ambient temperature to −150° C. at a rate of 25° C. per minute;
8. Decrease of ambient temperature to −160° C.;
9. Transfer to N₂ tank for long-term storage.

After low-temperature storage, the EN clusters were thawed and differentiated into islet-like clusters post-thaw according to the described procedures, with the combination differentiation protocol, with addition of GSI, without T3 treatment. Recovery rates and viability were determined as described above.

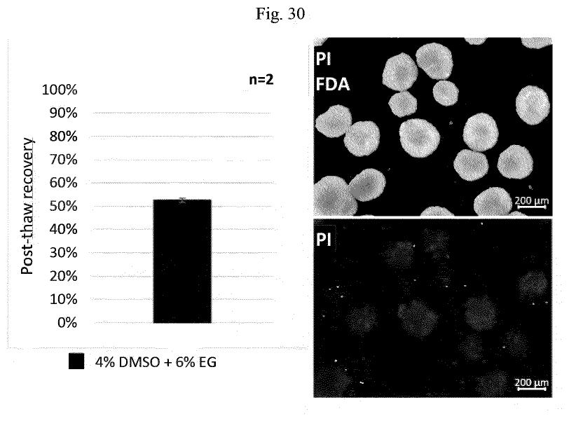

As shown in FIG. 30, application of the described freezing procedure and post-thaw differentiation in the presence of GSI and in the absence of T3 resulted in highly viable clusters with post-thaw recovery comparable to other protocols.

1. An in vitro method of producing an islet-like cluster, comprising the steps of
i) providing an endocrine progenitor cell cluster,
ii) differentiating the endocrine progenitor cell cluster to an islet-like cluster comprising an incubation step iia) of incubating the endocrine progenitor cell cluster in the presence of a gamma secretase inhibitor and the absence of thyroid hormone, and an incubation step iib) of incubating the cell cluster resulting from step iia) in the absence of a gamma secretase inhibitor and of thyroid hormone.

2. The method of claim 1, wherein step i) comprises
ia) providing a pluripotent cell cluster,
ib) differentiating the pluripotent cell cluster to a definitive endoderm cell cluster in the presence of a SMAD and MAPK signalling activator, a fibroblast growth factor and a polyanionic polymer,
ic) differentiating the definitive endoderm cell cluster to a gut tube cell cluster,
id) differentiating the gut tube cell cluster to a pancreatic progenitor cell cluster, and
ie) differentiating the pancreatic progenitor cell cluster to an endocrine progenitor cell cluster;
and optionally cryoprotecting the endocrine progenitor cell cluster; preferably according to the method of any one of claims 11-14.

3. The method of claim 1 or 2, wherein the endocrine progenitor cell cluster provided in step (i) is frozen and step (i) comprises thawing the frozen endocrine progenitor cell cluster; preferably thawing according to the method of claim 14.

4. A cell culture comprising a plurality of islet-like clusters, characterized in that 25% or less of the cells in the cell culture are C-peptide^{neg} and serotonin^{pos}; preferably wherein the islet-like clusters are obtainable by the method of any one of claims 1-3.

5. The cell culture of claim 4, further characterized in that 40% or more of the cells are C-peptide^{pos} and NKX6.1^{pos} beta cells.

6. The cell culture of claim 4 or 5, further characterized in that 25% or less, preferably 18% or less of the cells are VMAT1^{pos} cells.

7. The cell culture of any one of claims 4 to 6, further characterized in that 2% or less, preferably 1% or less, more preferably 0.6% or less of the cells are PDX1^{neg}/Ki67^{pos} cells.

8. The cell culture of any one of claims 4 to 7, further characterized in that there is no detectable level of CDX2^{pos}/VMAT1^{pos} cells.

9. A cell medium for differentiating an endocrine progenitor cell cluster towards an islet-like cluster, comprising a gamma secretase inhibitor and not comprising thyroid hormone.

10. A method of producing a definitive endoderm cell cluster, comprising the steps of
i) providing a pluripotent cell cluster,
ii) differentiating the pluripotent cell cluster to a definitive endoderm cell cluster as defined in step ib) of claim 2.

11. A method of producing a cryoprotected pancreatic lineage cell cluster, comprising the steps of
(i) providing a pancreatic lineage cell cluster, preferably an endocrine progenitor cell cluster within 1 to 3 days after reaching the endocrine stage, in a medium,
(ii) cryoprotecting the pancreatic lineage cell cluster by cooling the medium to a temperature of about 8° C. to about −6° C. in the presence of increasing concentrations of ethylene glycol (EG) and increasing concentrations of dimethylsulfoxide (DMSO), wherein the final concentration of EG is at least about 1% v/v and the final concentration of DMSO is at least about 1% v/v, and
(iii) optionally freezing the cryoprotected pancreatic lineage cell cluster to a temperature of at least about −40° C., thereby producing a frozen cryoprotected pancreatic lineage cell cluster.

12. The method of claim 11, wherein
the final concentration of EG is from about 5% to about 9% v/v, and/or the final concentration of DMSO is from about 3% to about 9% v/v, and/or
the final concentration of EG and DMSO combined is from about 7% to about 13% v/v.

13. The method of claim 11 or 12, wherein the final concentrations of EG and DMSO are obtained by steps comprising:
a) obtaining a first concentration of EG and DMSO and cooling the medium to a temperature from about 24° C. to about 18° C.;
b) obtaining a second concentration of EG and DMSO and cooling the medium to a temperature from about 8° C. to about −6° C.; and
c) obtaining a third concentration of EG and DMSO and maintaining the medium at the temperature of step b) or cooling the medium to a temperature from about 8° C. to about −6° C. that is lower than the temperature of step b).

14. The method of any one of claims 11 to 13, wherein the cryoprotected pancreatic lineage cell cluster is frozen to a temperature of at least about −40° C., preferably comprising the steps of:
1) cooling the medium to a temperature of about −5° C. to about −10° C. at a cooling rate avoiding ice nucleation,
2) incubating the medium at the temperature of step 1) until an even temperature distribution throughout the medium is achieved,
3) decreasing the ambient temperature to at least about −30° C. to induce ice nucleation,
4) increasing the ambient temperature to at least about −28° C. and maintaining the temperature until ice has propagated throughout the medium, and
5) cooling the medium to a temperature of at least about −40° C.

15. A cryoprotective medium comprising a pancreatic lineage cell cluster, preferably an endocrine progenitor cell cluster, wherein the cryoprotective medium comprises at least about 1% v/v EG and at least about 1% DMSO.

16. A frozen cell culture comprising a plurality of pancreatic lineage cell clusters, preferably endocrine progenitor cell clusters, wherein at least 20%, 30%, 40%, 50%, 60%, 70%, 80% or 90% of the pancreatic lineage cells are viable; preferably wherein the frozen cell culture is obtainable by the method of any one of claims 11 to 14.

17. A method of producing a thawed pancreatic lineage cell cluster, preferably a thawed endocrine progenitor cell cluster, comprising the steps of
(i) providing a frozen pancreatic lineage cell cluster obtainable by the method of any one of claims 11-14,
(ii) thawing the frozen pancreatic lineage cell cluster, preferably comprising contacting the pancreatic lineage cell cluster with a ROCK inhibitor, and
(iii) optionally differentiating the thawed cell cluster, preferably according to the method of any one of claims 1-3.

18. A thawed cell culture comprising a plurality of pancreatic lineage cell clusters, preferably endocrine progenitor cell clusters, wherein the thawed cell culture is obtained from a culture of frozen cells and the recovery of viable cells is at least 20% of the frozen cells; preferably wherein the thawed cell culture is obtainable by the method of claim 17.
